SEQUENCE LISTING (25 sequences)

SEQ ID NO: 1 - protein, 259 aa, Artificial sequence
  Synthetic modified mitochondrial DNA binding protein
MARRRRRRRRRRRMAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 2 - protein, 264 aa, Artificial Sequence
  Synthetic recombinant polypeptide
MRRRRRRRRRRRGEGDIMGEWGNEIFGAIAGFLGGEMLSRAVCGTSRQLPPVLGYLGSRQSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 3 - DNA, 795 bp, Artificial Sequence
  Synthetic nucleotide
atgcggcgacgcagacgtcgtcgtcggcggcgtcgcggcgagggtgatattatgggtgaatgggggaacgaaattttcggagcgatcgctggttttctcggtggagaaatgttatcacgcgcggtatgtggcaccagcaggcagctgcctccagtccttggctatctgggttcccgccagtcatcggtgttagcatcatgtccgaaaaaacctgtctcgtcgtacctgcgcttctccaaagagcagctgccgatttttaaagcgcaaaatccggatgctaaaacgactgaactgattcgccgcattgcacaacgctggcgcgaactcccggacagtaaaaaaaaaatttatcaggacgcctatcgggctgaatggcaggtctataaagaggagatctcacgcttcaaagaacaattaaccccgagtcaaataatgtctctggaaaaagaaatcatggataaacacttaaaacgaaaggcgatgacgaagaaaaaagaactgaccctgctaggtaaacctaagcgtccgcgctctgcgtataatgtgtacgtggcagaacgttttcaggaggccaaaggggattctccgcaagaaaaactgaagaccgtcaaagaaaattggaaaaacctgtctgatagcgaaaaagaactgtacattcagcacgctaaagaagatgagacgcggtatcacaacgaaatgaaatcttgggaagagcagatgatcgaggtcggtcggaaggatcttctccgtcgaaccatcaaaaaacagcgtaaatatggagcagaagagtgctga

SEQ ID NO: 4 - protein, 11 aa, Human immunodeficiency virus
YGRKKRRQRRR

SEQ ID NO: 5 - protein, 9 aa, Human immunodeficiency virus
RKKRRQRRR

SEQ ID NO: 6 - protein, 246 aa, Homo sapiens
MAFLRSMWGVLSALGRSGAELCTGCGSRLRSPFSFVYLPRWFSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 7 - protein, 7 aa, Unknown
  Protein transduction domain
RRRRRRR

SEQ ID NO: 8 - protein, 12 aa, Unknown
  PTD 5 (protein transduction domain)
RRQRRTSKLMKR

SEQ ID NO: 9 - protein, 27 aa, Artificial Sequence
  Transportan (Protein Transduction Domain)
GWTLNSAGYLLGKINLKALAALAKKIL

SEQ ID NO: 10 - protein, 30 aa, Artificial Sequence
  Protein Transduction Domain
AWEAKLAKALAKALAKHLAKALAKALKCEA

SEQ ID NO: 11 - protein, 16 aa, Unknown
  Protein Transduction Domain
RQIKIWFQNRRMKWKK

SEQ ID NO: 12 - protein, 7 aa, Polyomavirus sp.
PKKKRKV

SEQ ID NO: 13 - protein, 7 aa, Unknown
  Nucclear Localization Signal
GKKRSKV

SEQ ID NO: 14 - protein, 7 aa, Unknown
  Nuclear Localization Signal
KSRKRKL

SEQ ID NO: 15 - protein, 19 aa, Unknown
  Nuclear Localization Sigmal
KRPAATKKAGQAKKKKLDK

SEQ ID NO: 16 - protein, 20 aa, Unknown
  Nuclear Localization Signal
RKKRKTEEESPLKDKAKKSK

SEQ ID NO: 17 - protein, 20 aa, Unknown
  Nuclear Localization Signal
KDCVMNKHHRNRCQYCRLQR

SEQ ID NO: 18 - protein, 9 aa, Unknown
  Nuclear Localization Signal
PAAKRVKLD

SEQ ID NO: 19 - protein, 20 aa, Unknown
  Nuclear Localization Signal
KKYENVVIKRSPRKRGRPRK

SEQ ID NO: 20 - protein, 331 aa, Artificial Sequence
  Synthetic PTD-PA-TFAM
MRRRRRRRRRRRGEGDIMGEWGNEIFGAIAGFLGGEHDEAQQNAFYQVLNMPNLNADQRNGFIQSLKDDPSQSANVLGEAHDEAQQNAFYQVLNMPNLNADQRNGFIQSLKDDPSQSANVLGEAGEGSSVLASCPKKPVSSYLRFSKEQLPIFKAQNPDAKTTELIRRIAQRWRELPDSKKKIYQDAYRAEWQVYKEEISRFKEQLTPSQIMSLEKEIMDKHLKRKAMTKKKELTLLGKPKRPRSAYNVYVAERFQEAKGDSPQEKLKTVKENWKNLSDSEKELYIQHAKEDETRYHNEMKSWEEQMIEVGRKDLLRRTIKKQRKYGAEEC

SEQ ID NO: 21 - DNA, 10 bp, Unknown
  DNA binding site of SOX 9
agaacaatgg

SEQ ID NO: 22 - protein, 346 aa, Homo sapiens
MAASGKLSTCRLPPLPTIREIIKLLRLQAANELSQNFLLDLRLTDKIVRKAGNLTNAYVYEVGPGPGGITRSILNADVAELLVVEKDTRFIPGLQMLSDAAPGKLRIVHGDVLTFKVEKAFSESLKRPWEDDPPNVHIIGNLPFSVSTPLIIKWLENISCRDGPFVYGRTQMTLTFQKEVAERLAANTGSKQRSRLSVMAQYLCNVRHIFTIPGQAFVPKPEVDVGVVHFTPLIQPKIEQPFKLVEKVVQNVFQFRRKYCHRGLRMLFPEAQRLESTGRLLELADIDPTLRPRQLSISHFKSLCDVYRKMCDEDPQLFAYNFREELKRRKSKNEEKEEDDAENYRL

SEQ ID NO: 23 - protein, 396 aa, Homo sapiens
MWIPVVGLPRRLRLSALAGAGRFCILGSEAATRKHLPARNHCGLSDSSPQLWPEPDFRNPPRKASKASLDFKRYVTDRRLAETLAQIYLGKPSRPPHLLLECNPGPGILTQALLEAGAKVVALESDKTFIPHLESLGKNLDGKLRVIHCDFFKLDPRSGGVIKPPAMSSRGLFKNLGIEAVPWTADIPLKVVGMFPSRGEKRALWKLAYDLYSCTSIYKFGRIEVNMFIGEKEFQKLMADPGNPDLYHVLSVIWQLACEIKVLHMEPWSSFDIYTRKGPLENPKRRELLDQLQQKLYLIQMIPRQNLFTKNLTPMNYNIFFHLLKHCFGRRSATVIDHLRSLTPLDARDILMQIGKQEDEKVVNMHPQDFKTLFETIERSKDCAYKWLYDETLEDR

SEQ ID NO: 24 - protein, 1230 aa, Homo sapiens
MSALCWGRGAAGLKRALRPCGRPGLPGKEGTAGGVCGPRRSSSASPQEQDQDRRKDWGHVELLEVLQARVRQLQAESVSEVVVNRVDVARLPECGSGDGSLQPPRKVQMGAKDATPVPCGRWAKILEKDKRTQQMRMQRLKAKLQMPFQSGEFKALTRRLQVEPRLLSKQMAGCLEDCTRQAPESPWEEQLARLLQEAPGKLSLDVEQAPSGQHSQAQLSGQQQRLLAFFKCCLLTDQLPLAHHLLVVHHGQRQKRKLLTLDMYNAVMLGWARQGAFKELVYVLFMVKDAGLTPDLLSYAAALQCMGRQDQDAGTIERCLEQMSQEGLKLQALFTAVLLSEEDRATVLKAVHKVKPTFSLPPQLPPPVNTSKLLRDVYAKDGRVSYPKLHLPLKTLQCLFEKQLHMELASRVCVVSVEKPTLPSKEVKHARKTLKTLRDQWEKALCRALRETKNRLEREVYEGRFSLYPFLCLLDEREVVRMLLQVLQALPAQGESFTTLARELSARTFSRHVVQRQRVSGQVQALQNHYRKYLCLLASDAEVPEPCLPRQYWEELGAPEALREQPWPLPVQMELGKLLAEMLVQATQMPCSLDKPHRSSRLVPVLYHVYSFRNVQQIGILKPHPAYVQLLEKAAEPTLTFEAVDVPMLCPPLPWTSPHSGAFLLSPTKLMRTVEGATQHQELLETCPPTALHGALDALTQLGNCAWRVNGRVLDLVLQLFQAKGCPQLGVPAPPSEAPQPPEAHLPHSAAPARKAELRRELAHCQKVAREMHSLRAEALYRLSLAQHLRDRVFWLPHNMDFRGRTYPCPPHFNHLGSDVARALLEFAQGRPLGPHGLDWLKIHLVNLTGLKKREPLRKRLAFAEEVMDDILDSADQPLTGRKWWMGAEEPWQTLACCMEVANAVRASDPAAYVSHLPVHQDGSCNGLQHYAALGRDSVGAASVNLEPSDVPQDVYSGVAAQVEVFRRQDAQRGMRVAQVLEGFITRKVVKQTVMTVVYGVTRYGGRLQIEKRLRELSDFPQEFVWEASHYLVRQVFKSLQEMFSGTRAIQHWLTESARLISHMGSVVEWVTPLGVPVIQPYRLDSKVKQIGGGIQSITYTHNGDISRKPNTRKQKNGFPPNFIHSLDSSHMMLTALHCYRKGLTFVSVHDCYWTHAADVSVMNQVCREQFVRLHSEPILQDLSRFLVKRFCSEPQKILEASQLKETLQAVPKPGAFDLEQVKRSTYFFS

SEQ ID NO: 25 - DNA, 780 bp, Artificial Sequence
  Synthetic nucleic acid encoding modified mitochondrial DNA binding polypeptide
atggcgcgtcgtcgtcgtcgtcgtcgtcgtcgtcgtcgtatggcgtttctccgaagcatgtggggcgtgctgagtgccctgggaaggtctggagcagagctgtgcaccggctgtggaagtcgactgcgctcccccttcagttttgtgtatttaccgaggtggttttcatctgtcttggcaagttgtccaaagaaacctgtaagttcttaccttcgattttctaaagaacaactacccatatttaaagctcagaacccagatgcaaaaactacagaactaattagaagaattgcccagcgttggagggaacttcctgattcaaagaaaaaaatatatcaagatgcttatagggcggagtggcaggtatataaagaagagataagcagatttaaagaacagctaactccaagtcagattatgtctttggaaaaagaaatcatggacaaacatttaaaaaggaaagctatgacaaaaaaaaaagagttaacactgcttggaaaaccaaaaagacctcgttcagcttataacgtttatgtagctgaaagattccaagaagctaagggtgattcaccgcaggaaaagctgaagactgtaaaggaaaactggaaaaatctgtctgactctgaaaaggaattatatattcagcatgctaaagaggacgaaactcgttatcataatgaaatgaagtcttgggaagaacaaatgattgaagttggacgaaaggatcttctacgtcgcacaataaagaaacaacgaaaatatggtgctgaggagtgttaa

(19) United States
(12) Patent Application Publication
(10) Pub. No.: US 2009/0208478 A1
(43) Pub. Date: Aug. 20, 2009

(54) TRANSDUCIBLE POLYPEPTIDES FOR MODIFYING METABOLISM

(75) Inventor: Shaharyar M. Khan, Charlottesville, VA (US)

Correspondence Address: Pabst Patent Group LLP, 1545 PEACHTREE STREET NE, SUITE 320, ATLANTA, GA, 30309, US

(73) Assignee: Gencia Corporation

(21) Appl. No.: 12/390,225

(22) Filed: Feb. 20, 2009

Related U.S. Application Data
(63) Continuation-in-part of application No. 12/253,138, filed on Oct. 16, 2008.
Application No. 12/253,138 is a continuation-in-part of application No. 11/932,674, filed on Oct. 31, 2007.
Application No. 11/932,674 is a continuation-in-part of application No. 11/930,892, filed on Oct. 31, 2007.
Application No. 11/930,892 is a continuation-in-part of application No. 11/389,432, filed on Mar. 24, 2006.
Application No. 11/389,432 is a continuation-in-part of application No. 10/972,963, filed on Oct. 25, 2004.
(60) Provisional application No. 60/568,436, filed on May 5, 2004.
(60) Provisional application No. 60/513,983, filed on Oct. 24, 2003.

(51) Int. Cl. A61K 38/45; A61K 38/16; C07K 14/435; C12N 15/63

(52) U.S. Cl. 424/94.5; 514/12; 530/350; 435/320.1

(57) ABSTRACT

Methods and compositions for modifying the metabolism of a subject are provided. One embodiment provides a recombinant polypeptide having a polynucleotide-binding domain, a protein transduction domain, and a targeting domain. In a preferred embodiment, the polynucleotide-binding domain includes one or more HMG box domains.

### CROSS-REFERENCE TO RELATED APPLICATIONS

This application is a continuation-in-part application of U.S. patent application Ser. No. 12/253,138 filed on Oct. 16, 2008, which is a continuation in part of U.S. patent application Ser. No. 11/932,674 filed on Oct. 31, 2007, and is a continuation-in-part of U.S. patent application Ser. No. 11/930,892, filed on Oct. 31, 2007, and is a continuation-in-part of U.S. patent application Ser. No. 11/389,432, filed on Mar. 24, 2006, which is a continuation-in-part of U.S. patent application Ser. No. 10/972,963 filed on Oct. 25, 2004, which claims priority to U.S. Provisional Application No. 60/568,436 filed on May 5, 2004, and U.S. Provisional Application No. 60/513,983 filed on Oct. 24, 2003, and where permissible each of which is incorporated by reference in their entirety.

### STATEMENT REGARDING FEDERALLY SPONSORED RESEARCH OR DEVELOPMENT

Aspects of the following disclosure were supported in part by grant number AG023443-02 awarded by the National Institutes of Health. Therefore, the United States has certain rights in the disclosure.

### FIELD OF THE INVENTION

The present disclosure is generally directed to compositions and methods for the delivery of transducible DNA binding proteins to organelles, more particularly to compositions and methods for modifying mitochondria, for example inducing mitochondrial biogenesis and oxidative metabolism.

### BACKGROUND

In the course of evolution, many organisms tackled the task of introducing macromolecules into living cells. Aside from the cell-specific, usually receptor-mediated or active uptake mechanisms, the general solution that has independently emerged in many lineages relies on peptides specifically evolved to interact with, and insert into lipid bilayer membranes. Thus, bacterial colicins, human porins, and protein transduction domains (PTDs) from diverse species share the motif of a positively charged alpha-helix, frequently with an amphipathic structure, which is capable of inserting into lipid membranes, and delivering larger cargoes intracellularly. Recent research reports confirm the successful use of PTDs fused to proteins for their delivery across biological boundaries, including the blood-brain barrier, and the placenta.

Mitochondria are the α-proteobacterial, energy producing and cell death controlling endosymbionts in all eukaryotic cells (Fitzpatrick, et al., Mol Biol Evol., 23(1):74-85 (2006)). The mitochondrial outer membrane has a lipid composition similar to that of eukaryotic plasma membranes, whereas the mitochondrial inner membrane contains a unique lipid, cardiolipin, and more closely resembles bacterial membranes. Mitochondria contain their own DNA, transcription and translation machinery, involved in producing proteins necessary to carry out oxidative phosphorylation. Many diseases are associated with a decline in mitochondrial number, mitochondrial function, or more specifically, oxidative phosphorylation. Furthermore, increasing mitochondrial function, number and/or oxidative phosphorylation in a basal or healthy state may increase the capabilities of cells and tissues heavily reliant on mitochondrial metabolism, such as brain and muscle.

Therefore it is an object of the invention to provide compositions and methods for increasing mitochondrial number, mitochondrial function and/or oxidative phosphorylation in a subject.

### SUMMARY

Compositions and methods for increasing mitochondrial biological activity as well as numbers of mitochondria are provided. One embodiment provides compositions containing one or more mitochondrial DNA (mtDNA) binding proteins in an amount effective to increase mitochondrial biological activity, for example mitochondrial respiration or biogenesis. Preferred mitochondrial DNA binding proteins include, but are not limited to mitochondrial transcription factors such as transcription factor A, mitochondrial (TFAM); transcription factor B1, mitochondrial (TFB1M); transcription factor B2, mitochondrial (TFB2M); and variants thereof. Additional mitochondrial DNA binding proteins that can be used to increase mitochondrial biological activity include proteins involved in mtDNA replication and translation such as polymerase (RNA) mitochondrial (DNA directed) (POLRMT).

Another embodiment provides a recombinant polypeptide including a mitochondrial transcription factor, a protein transduction domain operably linked to the mitochondrial transcription factor, and a non-nuclear localization signal operably linked to mitochondrial transcription factor. In a preferred embodiment, the recombinant polypeptide is encoded by a nucleic acid having at least 95% sequence identity to SEQ ID NO:3.

Another embodiment provides a pharmaceutical composition including a recombinant polypeptide having a mitochondrial transcription factor A (TFAM) polypeptide or a variant thereof wherein the TFAM variant includes one or more substitutions, additions, deletions relative to the TFAM polypeptide, and a protein transduction domain operably linked to the TFAM polypeptide or variant thereof, and a non-nuclear localization signal operably linked to the TFAM polypeptide or variant thereof, and a pharmaceutically acceptable carrier.

Yet another embodiment provides a recombinant polypeptide having an amino acid sequence including amino acid number 63 to amino acid number 128 of SEQ ID NO: 1, a protein transduction domain operably linked to the recombinant polypeptide; and a non-nuclear localization signal operably linked to the recombinant polypeptide. The recombinant polypeptide can further include an amino acid sequence comprising amino acid number 168 to amino acid number 229 of SEQ ID NO: 1.

Another recombinant polypeptide includes an amino acid sequence starting at amino acid number 168 to amino acid number 229 of SEQ ID NO: 1, a protein transduction domain operably linked to the recombinant polypeptide, and a non-nuclear localization signal operably linked to the recombinant polypeptide.

Other a recombinant polypeptides include at least one HMG box domain wherein the HMG box domain has 75 amino acid residues, and wherein the 75 amino acid residues include highly conserved proline, aromatic and basic residues, a protein transduction domain operably linked to the recombinant polypeptide, and a non-nuclear localization signal operably linked to the recombinant polypeptide.

A non-viral vector is also provided. The non-viral vector includes a recombinant polypeptide with at least two HMG box domains wherein the HMG box domains comprise 75 amino acid residues wherein the 75 amino acid residues include highly conserved proline, aromatic and basic residues, a protein transduction domain operably linked to the recombinant polypeptide, and a non-nuclear localization signal operably linked to the recombinant polypeptide.

Methods for treating mitochondrial diseases using the disclosed polypeptides are also provided. The methods include administering to a subject an effective amount of the disclosed recombinant polypeptides to alleviate a symptom of the mitochondrial disease.

Still another embodiment provides modified polypeptides having a polynucleotide-binding domain, a targeting domain, for example an organelle specific targeting signal, and a protein transduction domain. In certain embodiments, the modified polypeptide has at least one HMG box domain, more typically at least two HMG box domains. The polypeptide is designed to enter cells and target specific organelles, such as mitochondria. The targeting signal helps direct the polypeptide to a site of interest, for example an organelle and thereby modify the function of the cellular organelle.

In a preferred embodiment, the recombinant polypeptide has at least 80, 85, 90, 95, 97, 99, or 100% sequence identity to

(SEQ ID NO: 2)

MRRRRRRRRR RRGEGDIMGE WGNEIFGAIA GFLGGEMLSR
AVCGTSRQLP PVLGYLGSRQSSVLASCPKK PVSSYLRFSK
EQLPIFKAQN PDAKTTELIR RIAQRWRELP
DSKKKIYQDAYRAEWQVYKE EISRFKEQLT PSQIMSLEKE
IMDKHLKRKA MTKKKELTLL GKPKRPRSAYNVYVAERFQE
AKGDSPQEKL KTVKENWKNL SDSEKELYIQ HAKEDETRYH
NEMKSWEEQMIEVGRKDLLR RTIKKQRKYG AEEC

In another embodiment, the recombinant polypeptide is encoded by a nucleic acid having at least 80, 85, 90, 95, 97, 99, or 100% sequence identity to

(SEQ ID NO:3)

atgcggcgac gcagacgtcg tcgtcggcgg cgtcgcggcg
agggtgatat tatgggtgaa tgggggaacg aaattttcgg
agcgatcgct ggttttctcg gtggagaaat gttatcacgc
gcggtatgtg gcaccagcag gcagctgcct ccagtccttg
gctatctggg ttcccgccag tcatcggtgt tagcatcatg
tccgaaaaaa cctgtctcgt cgtacctgcg cttctccaaa
gagcagctgc cgatttttaa agcgcaaaat ccggatgcta
aaacgactga actgattcgc cgcattgcac aacgctggcg
cgaactcccg gacagtaaaa aaaaaattta tcaggacgcc
tatcgggctg aatggcaggt ctataaagag gagatctcac
gcttcaaaga acaattaacc ccgagtcaaa taatgtctct
ggaaaaagaa atcatggata aacacttaaa acgaaaggcg
atgacgaaga aaaaagaact gaccctgcta ggtaaaccta
agcgtccgcg ctctgcgtat aatgtgtacg tggcagaacg
ttttcaggag gccaaagggg attctccgca agaaaaactg
aagaccgtca aagaaaattg gaaaaacctg tctgatagcg
aaaaagaact gtacattcag cacgctaaag aagatgagac
gcggtatcac aacgaaatga aatcttggga agagcagatg
atcgaggtcg gtcggaagga tcttctccgt cgaaccatca
aaaaacagcg taaatatgga gcagaagagt gctga

Another embodiment provides compositions and methods of inducing mitochondrial biogenesis and increasing mitochondrial oxidative metabolism by administering an effective amount of a recombinant polypeptide having a polynucleotide-binding domain, a targeting domain, and a protein transduction domain to a cell to increase mitochondrial biogenesis relative to a control, One embodiment provides a pharmaceutical composition consisting essentially of a recombinant polypeptide having a polynucleotide-binding domain, a targeting domain, and a protein transduction domain and a pharmaceutically acceptable carrier or excipient. Preferably the polynucleotide-binding domain includes TFAM or a fragment thereof capable of binding a polynucleotide. Another embodiment provides a method for reducing body weight by administering an effective amount of the disclosed polypeptides to a subject to increase mitochondrial oxidative metabolism in the subject.

A preferred embodiment provides a fusion protein having three polypeptide regions or domains. The first region is the N-terminus region and includes a PTD. The PTD is operably linked to the second region which includes a targeting signal or domain. The second region is operably linked to a third region including a polypeptide that binds to or condenses mitochondrial DNA. Preferably, the polynucleotide-binding polypeptide is a mitochondrial transcription factor. A preferred polypeptide that binds or condenses mitochondrial DNA includes, but is not limited to TFAM or a fragment thereof capable of binding or condensing mitochondrial DNA or of promoting or inducing transcription of mitochondrial DNA.

### BRIEF DESCRIPTION OF THE DRAWINGS

FIGS. 1A-C are bar graphs showing the effect of recombinant TFAM on basal respiration of LHON cybrid cells. FIG. 1A shows percent of control basal respiration versus days after treatment with recombinant TFAM. FIG. 1B shows percent of control mean mtDNA gene copy number versus days after treatment with recombinant TFAM. FIG. 1C shows percent of control mean mtRNA (cDNA) copy number.

FIG. 2 depicts a bar graph of percent buffer control of the mitofilin/b-actin, CI, CII, CIII, CIV, and CV versus days after treatment with recombinant TFAM.

FIG. 3A shows a bar graph of percent buffer control of oxidative metabolism in brain, heart, liver, and skeletal muscle. FIG. 3B shows a bar graph of TFAM/control versus mtDNA gene cDNA.

FIG. 4 shows a bar graph of body weight (grams) in mice treated with vehicle, recombinant TFAM, vehicle for 3 months, and recombinant TFAM over 3 months.

FIG. 5 shows a bar graph of percent food intake in mice injected with recombinant TFAM or vehicle over three months.

FIG. 6 shows a bar graph of oxygen uptake in cells treated with recombinant TFAM or vehicle.

FIG. 7 shows a bar graph of percent invasive index in tumor cells treated with recombinant TFAM or vehicle assayed after twenty-four hours.

FIG. 8 shows a bar graph of electron transport activities of mitochondria from mouse brain in mice injected with recombinant TFAM or vehicle with and without exercise.

### DETAILED DESCRIPTION

### 1. Definitions

In describing and claiming the disclosed subject matter, the following terminology will be used in accordance with the definitions set forth below.

The term “polypeptides” includes proteins and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

“Variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered, The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues, arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gin: Asn), (Glu: Asp), (Gly: Ala), (His: Asn, Gln), (lie: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

“Identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, the term “low stringency” refers to conditions that permit a polynucleotide or polypeptide to bind to another substance with little or no sequence specificity.

As used herein, the term “purified” and like terms relate to the isolation of a molecule or compound in a form that is substantially free (at least 60% free, preferably 75% free, and most preferably 90% free) from other components normally associated with the molecule or compound in a native environment.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the term “treating” includes alleviating the symptoms associated with a specific disorder or condition and/or preventing or eliminating said symptoms.

“Operably linked” refers to a juxtaposition wherein the components are configured so as to perform their usual function. For example, control sequences or promoters operably linked to a coding sequence are capable of effecting the expression of the coding sequence, and an organelle localization sequence operably linked to protein will assist the linked protein to be localized at the specific organelle.

“Localization Signal or Sequence or Domain” or “Targeting Signal or Sequence or Domain” are used interchangeably and refer to a signal that directs a molecule to a specific cell, tissue, organelle, intracellular region or cell state. The signal can be polynucleotide, polypeptide, or carbohydrate moiety or can be an organic or inorganic compound sufficient to direct an attached molecule to a desired location. Exemplary targeting signals include cell targeting signals known in the art such as those provided in Table 1 and described in Wagner et al., Adv Gen, 53:333-354 (2005) the disclosures of which are incorporated herein by reference in their entirety. It will be appreciated that the entire sequence listed in Table 1 need not be included, and modifications including truncations of these sequences are within the scope of the disclosure provided the sequences operate to direct a linked molecule to a specific cell type. Targeting signals of the present disclosure can have 80 to 100% sequence identity to the sequences in Table 1. One class of suitable targeting signals include those that do not interact with the targeted cell in a receptor:ligand mechanism. For example, targeting signals include signals having or conferring a net charge, for example a positive charge. Positively charged signals can be used to target negatively charged cell types such as neurons and muscle. Negatively charged signals can be used to target positively charged cells.

“Tropism” refers to the propensity of a molecule to be attracted to a specific cell, cell type or cell state. In the art, tropism can refer to the way in which different viruses and pathogens have evolved to preferentially target to specific host species, or specific cell types within those species. The propensity for a molecule to be attracted to a specific cell, cell type or cell state can be accomplished by means of a targeting signal.

“Cell Type” is a manner of grouping or classifying cells in the art. The term cell type refers to the grouping of cells based on their biological character determined in part through common biological function, location, morphology, structure, expression of polypeptides, nucleotides or metabolites.

“Cell State” refers to the condition of a cell type. Cells are dynamic throughout their life and can achieve various states of differentiation, function, morphology and structure. As used herein, cell state refers to a specific cell type throughout its lifetime.

As used herein, the term “cell surface marker” refers to any molecule such as moiety, peptide, protein, carbohydrate, nucleic acid, antibody, antigen, and/or metabolite presented on the surface or in the vicinity of a cell sufficient to identify the cell as unique in either type or state.

“Protein Transduction Domain” or PTD refers to a polypeptide, polynucleotide, carbohydrate, or organic or inorganic compounds that facilitates traversing a lipid bilayer, micelle, cell membrane, organelle membrane, or vesicle membrane, A PTD attached to another molecule facilitates the molecule traversing membranes, for example going from extracellular space to intracellular space, or cytosol to within an organelle. Exemplary PTDs include but are not limited to HIV TAT YGRKKRRQRRR (SEQ. ID. NO.:4) or RKKRRQRRR (SEQ, ID. NO.: 5); 11 Arginine residues, or positively charged polypeptides or polynucleotides having 8-15 residues, preferably 9-11 residues.

### 2. Mitochondrial DNA-binding Polypeptides

Compositions for modifying mitochondrial function in a subject are provided. In particular, the disclosed compositions cause an increase in mitochondrial number and/or an increase in mitochondrial respiration relative to a control. Preferred compositions include a mitochondrial DNA-binding polypeptide including, but not limited to mitochondrial transcription factors such as transcription factor A, mitochondrial (TFAM) having GenBank Accession No. mitochondrial NM 003201; transcription factor B1, mitochondrial (TFB1M) having GenBank Accession No. AF151833; transcription factor B2, mitochondrial (TFB2M) having GenBank Accession No. AK026835; and variants thereof.

A. Polynucleotide Binding Domain

The compositions and methods for the delivery of a polypeptide, for example a DNA-binding protein, provided herein include polynucleotide-binding polypeptides or polynucleotide-packaging polypeptides optionally having a PTD and optionally having a targeting signal or domain. The mitochondrial DNA-binding polypeptide can be any polypeptide known to bind or package a mtDNA. Preferably, the mtDNA-binding polypeptide is a recombinant polypeptide. The recombinant polypeptide can be used as a therapeutic agent either alone or in combination with a polynucleotide encoding a mitochondrial polypeptide. In one embodiment, the mtDNA-binding polypeptide includes at least a portion of a member of the high mobility group (HMG) of proteins to bind a polynucleotide, in particular at least one HMG box domain.

1. HMG Domain

Generally, the HMS domain includes a global fold of three helices stabilized in an ‘L-shaped’ configuration by two hydrophobic cores, The high mobility group chromosomal proteins HMG1 or HMG2, which are common to all eukaryotes, bind DNA in a non-sequence-specific fashion, for example to promote chromatin function and gene regulation. They can interact directly with nucleosomes and are believed to be modulators of chromatin structure. They are also important in activating a number of regulators of gene expression, including p53, Hox transcription factors and steroid hormone receptors, by increasing their affinity for DNA. HMG proteins include HMG-1/2, HMG-I(Y) and HMG-14/17.

The HMCG-1/2-box proteins can be further distinguished into three subfamilies according to the number of HMG domains present in the protein, their specific of sequence recognition and their evolutionary relationship. The first group contains chromosomal proteins bound to DNA with no sequence specificity (class I, HMG1 and HMG2), the second contains ribosomal and mitochondrial transcription factors which show sequence specificity in the presence of another associating factor when bound with DNA (class II, yeast ARS binding protein ABF-2, UBF and mitochondrial transcription factor mtTF-1), and the third contains gene-specific transcription factors which show sequence specific DNA binding (class III, lymphoid enhancer-binding factors LEF-1 and TCF-1; the mammalian sex-determining factor SRY, and the closely related SOX proteins; and the fungal regulatory proteins Mat-MC, Mat-a1, Ste11 and Rox1). The HMG 1/2-box DNA binding domain is about 75 to about 80 amino acids and contains highly conserved proline, aromatic and basic residues. Common properties of HMG domain proteins include interaction with the minor groove of the DNA helix, binding to irregular DNA structure, and the capacity to modulate DNA structure by bending.

SOX (SRY-type HMG box) proteins have critical functions in a number of developmental processes, including sex determination, skeleton formation, pre-B and T cell development and neural induction. SOX9 plays a direct role during chondrogenesis by binding and activating the chondrocyte-spacific enhancer of the Col2a1 gene. Loss of SOX9 gene function leads to the genetic condition known as Campomelic Dysplsia (CD), a form of dwarfism characterized by extreme skeletal malformation, and one in which three-quarters of XY individual are either intersexes or exhibit male to female sex reversal. There are more than 20 members cloned in SOX family. All of which contain an HMG domain, which can bind specifically to the double strand DNA motif and shares >50% identify with the HMG domain of SRY, the human testis-determining factor. The preferred DNA-binding site of SOX9 have been defined to be AGAACAATGG (SEQ ID NO: 21), which contains the SOX core-binding element (SCBE), AACAAT, flanking 5′ AG and 3′ GG nucleotides enhance binding by SOX9.

In one embodiment, the recombinant mtDNA-binding protein has at least one HMG box domain, generally at least two, more particularly 2-5 HMG box domains. The HMG box domain can bind to an AT rich DNA sequence, for example, using a large surface on the concave face of the protein, to bind the minor groove of the DNA. This binding bends the DNA helix axis away from the site of contact. The first and second helices contact the DNA, their N-termini fitting into the minor groove whereas helix 3 is primarily exposed to solvent. Partial intercalation of aliphatic and aromatic residues in helix 2 occurs in the minor groove.

In other embodiments, the mtDNA binding polypeptide can have at least one polynucleotide binding domain, typically two or more polynucleotide binding domains. The polynucleotide binding domains can be the same or different. For example, the polynucleotide-binding polypeptide can include at least one HMG box in combination with one or more DNA binding domains selected from the group consisting of an HMG box, homeodomain and POU domain; zinc finger domain such as C₂H₂ and C₂C₂; amphipathic helix domain such as leucine zipper and helix-loop-helix domains; and histone folds. The polynucleotide binding domain can be specific for a specific polynucleotide sequence, or preferably non-specifically binds to a polynucleotide. Alternatively, the polynucleotide-binding polypeptide can have more a combination of at least one polynucleotide binding domain that binds in a sequence specific manner and at least one polynucleotide binding-domain that binds DNA non-specifically.

2. Helix-turn-Helix

Certain embodiments provide mtDNA-binding polypeptides having a helix-turn-helix motif or at least a polynucleotide binding region of a helix-turn-helix protein, Helix-turn-helix proteins have a similar structure to bacterial regulatory proteins such as the I repressor and cro proteins, the lac repressor and so on which bind as dimers and their binding sites are palindromic. They contain 3 a helical regions separated by short turns which is why they are called helix-turn-helix proteins. One protein helix (helix 3) in each subunit of the dimer occupies the major groove of two successive turns of the DNA helix. Thus, in another embodiment, the disclosed polynucleotide-binding polypeptides can form dimers or other multi-component complexes, and have 1 to 3 helices.

3. Homeodomain

In yet another embodiment, the mtDNA-binding polypeptide includes a homeodomain or a portion of a homeodomain protein. Homeodomain proteins bind to a sequence of 180 base pairs initially identified in a group of genes called homeotic genes. Accordingly, the sequence was called the homeobox. The 180 bp corresponds to 60 amino acids in the corresponding protein. This protein domain is called the homeodomain. Homeodomain-containing proteins have since been identified in a wide range of organisms including vertebrates and plants. The homeodomain shows a high degree of sequence conservation. The homeodomain contains 4 a helical regions. Helices II and III are connected by 3 amino acids comprising a turn. This region has a very similar structure to helices II and III of bacterial DNA binding proteins.

4. Zinc Finger

Yet another embodiment provides a modified mtDNA-binding polypeptide having a zinc finger domain or at least a portion of a zinc finger protein. Zinc finger proteins have a domain with the general structure: Phe (sometimes Tyr)—Cys—2 to 4 amino acids—Cys—3 amino acids—Phe (sometimes Tyr)-5 amino acids—Leu—2 amino acids—His—3 amino acids—His, The phenylalanine or tyrosine residues which occur at invariant positions are required for DNA binding. Similar sequences have been found in a range of other DNA binding proteins though the number of fingers varies. For example, the SP1 transcription factor which binds to the GC box found in the promoter proximal region of a number of genes has 3 fingers. This type of zinc finger which has 2 cysteines and 2 histidines is called a C₂H₂ zinc finger.

Another type of zinc finger which binds zinc between 2 pairs of cysteines has been found in a range of DNA binding proteins. The general structure of this type of zinc finger is: Cys—2 amino acids—Cys—13 amino acids—Cys—2 amino acids—Cys. This is called a C₂C₂ zinc finger. It is found in a group of proteins known as the steroid receptor superfamily, each of which has 2 C₂C₂ zinc fingers.

5. Leucine zipper

Another embodiment provides a modified mtDNA-binding polypeptide having a leucine zipper or at least a portion of a leucine zipper protein. The first leucine zipper protein was identified from extracts of liver cells, and it was called C/EBF because it is an enhancer binding protein and it was originally thought to bind to the CAAT promoter proximal sequence. C/EBP will only bind to DNA as a dimer. The region of the protein where the two monomers join to make the dimer is called the dimerization domain. This lies towards the C-terminal end of the protein. When the amino acid sequence was examined it was found that a leucine residue occurs every seventh amino acid over a stretch of 35 amino acids. If this region were to form an a helix then all of these leucines would align on one face of the helix.

Because leucine has a hydrophobic side chain, one face of the helix is very hydrophobic. The opposite face has amino acids with charged side chains which are hydrophilic. The combination of hydrophobic and hydrophilic characteristics gives the molecule is amphipathic moniker. Adjacent to the leucine zipper region is a region of 20-30 amino acids which is rich in the basic (positively charged) amino acids lysine and arginine. This is the DNA binding domain—often referred to as the bZIP domain—the basic region of the leucine zipper. C/EBP is thought to bind to DNA by these bZIP regions wrapping round the DNA helix

The leucine zipper—bZIP structure has been found in a range of other proteins including the products of the jun and fos oncogenes. Whereas C/EBP binds to DNA as a homodimer of identical subunits, fos cannot form homodimers at all and jun/jun homodimers tend to be unstable. However fos/jun heterodimers are much more stable. These fos/jun heterodimers correspond to a general transcription factor called AP1 which binds to a variety of promoters and enhancers and activates transcription. The consensus API binding site is TGACTCA which is palindromic.

6. Helix-loop-Helix

Another embodiment provides a modified mtDNA-binding polypeptide having helix-loop-helix domain or a polynucleotide binding portion of a helix-loop-helix protein. Helix-loop-helix proteins are similar to leucine zippers in that they form dimers via amphipathic helices. They were first discovered as a class of proteins when a region of similarity was noticed between two enhancer binding proteins called E47 and EL12. This conserved region has the potential to form two amphipathic separated by a loop hence helix-loop-helix. Next to the dimerization domain is a DNA binding domain, again rich in basic amino acids and referred to as the bHLH domain. These structures are also found in a number of genes required for development of the Drosophila nervous system—the Achaete-scute complex, and in a protein called MyoD which is required for mammalian muscle differentiation.

7. Histone Fold

In still another embodiment, the modified mtDNA-binding polypeptide includes a histone polypeptide, a fragment of a histone polypeptide, or at least one histone fold. Histone folds exist in histone polypeptides monomers assembled into dimers. Histone polypeptides include H2A, H₂B, H3, and H4 which can form heterodimers H2A-2B and H3-H4. It will be appreciated that histone-like polypeptides can also be used in the disclosed compositions and methods. Histone-like polypeptides include, but are not limited to, HMf or the histone from Methanothermous fervidus, other archaeal histones known in the art, and histone-fold containing polypeptides such as MJ 1647, CBF, TAFII or transcription factor IID, SPT3, and Dr1-DRAP (Sanderman, K., et al., Cell. Mol Life Sci. 54:1350-1364 (1998), which is incorporated by reference in its entirety).

B. Transcription Factor A, Mitochondria (TFAM)

One embodiment, among others, provides a non-histone mtDNA-binding polypeptide, for example mitochondrial transcription factor A (TFAM) polypeptide, a variant thereof, or a fragment thereof sufficient to bind mtDNA. Variant TFAM can have 80%, 85%, 90%, 95%, 99% or greater sequence identity with a reference TFAM, for example naturally occurring TFAM having GenBank Accession No. NM_{—}003201.

TFAM is a member of the high mobility group (HMG) of proteins having two HMG-box domains. TFAM as well as other HMG proteins bind, wrap, bend, and unwind DNA, Thus, embodiments of the present disclosure include polynucleotide binding polypeptides including one or more polynucleotide binding regions of the HMG family of proteins, and optionally induce a structural change in the polynucleotide when the polypeptide binds or becomes associated with the polynucleotide. By inducing a conformational change in the polynucleotide, the polypeptide packages the polynucleotide. It has been reported that TFAM binds to mitochondrial DNA in a ratio of 900:1 (Alam, T. I., et al., Nucleic Acid Res. 31(6):1640-1645 (2003)). It will be appreciated that the amount of polynucleotide-binding polypeptide used in the compositions and methods disclosed herein can vary depending on the size and amount of the polynucleotide to be delivered. Suitable ratios of polynucleotide-binding polypeptide to base pairs of polynucleotide to be delivered include, but are not limited to, about 1:1 to 1:1,000; more preferably 1:100; even more preferably 1: about 10 to about 20 base pairs of polynucleotide to be delivered. It will also be appreciated that TFAM, another mtDNA-binding polypeptide, or a combination of two or more mtDNA-binding polypeptides can be added to a polynucleotide, preferably mtDNA, to wrap or cover the polynucleotide, and thereby package the polynucleotide and protected it from degradation.

A preferred TFAM polypeptide has at least 80, 85, 90, 95, 99, or 100 percent sequence identity to

(SEQ ID NO:6)

MAFLRSMWGVLSALGRSGAELCTGCGSRLRSPFSFVYLPRWFSSVLASCP
KKPVSSYLRFSKEQLPIFKAQNPDAKTTELIRRIAQRWRELPDSKKKIYQ
DAYRAEWQVYKEEISRFKEQLTPSQIMSLEKEIMDKHLKRKAMTKKKELT
LLGKPKRPRSAYNVYVAERFQEAKGDSPQEKLKTVKENWKNLSDSEKELY
IQHAKEDETRYKNEMKSWEEQMIEVGRKDLLRRTIKKQRKYGAEEC.

Variants of TFAM are also provided. One embodiment provides a TFAM polypeptide having one or more serine residues at positions 1, 2 and 13 substituted with a alanine or threonine residue. A preferred embodiment provides a TFAM polypeptide having serine 13 substituted for an alanine or threonine. The variant TFAM polypeptides have improved mtDNA binding in the presence of glucose or elevated glucose levels.

C. Transcription Factor B1, Mitochondrial (TFBIM)

The mtDNA-binding polypeptide can be transcription factor B1, mitochondrial (TFB1M). A preferred TFBIM has GenBanic Accession No. AF151833. TFB1 is part of the complex involved in mitochondrial transcription. The process of transcription initiation in mitochondria involves three types of proteins: the mitochondrial RNA polymerase (POLRMT), mitochondrial transcription factor A (TFAM), and mitochondrial transcription factors B1 and B2 (TFB1M, TFB2M). POLRMT, TFAM, and TFB1M or TFB2M assemble at the mitochondrial promoters and begin transcription. TFB1M has about 1/10 the transcriptional activity of TFB2M, and both TFBs are also related to rRNA methyltransferases and TFB1M can bind S-adenosylmethionine and methylate mitochondrial 12S rRNA. Additionally, TFB1M and TFB2M can bind single-stranded nucleic acids.

A preferred TFB1M polypeptide has at least 80, 85, 90, 95, 99, or 100 percent sequence identity to

(SEQ ID NO:22)

MAASGKLSTCRLPPLPTIREIIKLLRLQAANELSQNFLLDLRLTDKIVRK
AGNLTNAYVYEVGPGPGGITRSILNADVAELLVVEKDTRFIPGLQMLSDA
APGKLRIVHGDVLTFKVEKAFSESLKRPWEDDPPNVHIIGNLPFSVSTPL
IIKWLENISCRDGPFVYGRTQMTLTFQKEVAERLAANTGSKQRSRLSVMA
QYLCNVRHIFTIIPGQAFVPKPEVDVGVVHFTPLIQPKIEQPFKLVEKVV
QNVFQFRRKYCHRGLRMLFPEAQRLESTGRLLELADIDPTLRPRQLSISH
FKSLCDVYRKMCDEDPQLFAYNFREELKRRKSKNEEKEEDDAENYRL.

D. Transcription Factor B2, Mitochondrial (TFB2M)

In still another embodiment, the mtDNA-binding polypeptide includes TFB2M. In a preferred embodiment the TFB2M polypeptide has GenBank Accession No. AK026835. TFB2M also possesses a Rossmann-fold making it part of the NAD-binding protein family. TFB2M levels modulate mtDNA copy number and levels of mitochondrial transcripts as would be expected of a mitochondrial transcription factor. It is appreciated by those skilled in the art that increased activity of mitochondria causes an increase in mitochondrial biogenesis.

A preferred TFB2M polypeptide has at least 80, 85, 90, 95, 99, or 100 percent sequence identity to

(SEQ ID NO:23)

MWIPVVGLPRRLRLSALAGAGRFCILGSEAATRKHLPARNHCGLSDSSPQ
LWPEPDFRNPPRKASKASLDFKRYVTDRRLAETLAQIYLGKPSRPPHLLL
ECNPGPGILTQALLEAGAKVVALESDKTFIPHLESLGKNLDGKLRVIHCD
FFKLDPRSGGVIKPPAMSSRGLFKNLGIEAVPWTADIPLKVVGMFPSRGE
KRALWKLAYDLYSCTSIYKFGRIEVNMFIGEKEFQKLMADPGNPDLYHVL
SVIWQLACEIKVLHMEPWSSFDIYTRKGPLENPKRRELLDQLQQKLYLIQ
MIPRQNLFTKNLTPMNYNIFFHLLKHCFGRRSATVIDHLRSLTPLDARDI
LMQIGKQLDEKVVNMHPQDFKTLFETIERSKDCAYKWLYDETLEDR.

E. Polymerase (RNA) Mitochondrial (DNA Directed) (POLRMT)

Still another mtDNA binding polypeptide that can be used to modulate mitochondrial biological activity is POLRMT. In a preferred embodiment, the POLRMT polypeptide has GenBank Accession No. NM_{—}005035. POLRMT is a mitochondrial RNA polymerase similar in structure to phage RNA polymerases. Unlike phage polymerases, POLRMT contains two pentatricopeptide repeat (PPR) domains involved in regulating mitochondrial transcripts. It is appreciated by those skilled in the art that deletion of regulatory domains enables constitutive function.

A preferred POLRMT polypeptide has at least 80, 85, 90, 95, 99, or 100 percent sequence identity to

(SEQ ID NO:24)

MSALCWGRGAAGLKRALRPCGRPGLPGKEGTAGGVCGPRRSSSASPQEQD
QDRRKDWGHVELLEVLQARVRQLQAESVSEVVVNRVDVARLPECGSGDGS
LQPPRKVQMGAKDATPVPCGRWAKILEKDKRTQQMRMQRLKAKLQMPFQS
GEFKALTRRLQVEPRLLSKQMAGCLEDCTRQAPESPWEEQLARLLQEAPG
KLSLDVEQAPSGQHSQAQLSGQQQRLLAFFKCCLLTDQLPLAHHLLVVHH
GQRQKRKLLTLDMYNAVMLGWARQGAFKELVYVLFMVKDAGLTPDLLSYA
AALQCMGRQDQDAGTIERCLEQMSQEGLKLQALFTAVLLSEEDRATVLKA
VHKVKPTFSLPPQLPPPVNTSKLLRDVYAKDGRVSYPKLHLPLKTLQCLF
EKQLHMELASRVCVVSVEKPTLPSKEVKHARKTLKTLRDQWBEALCRALR
ETKNRLEREVYLGRFSLYPFLCLLDEREVVRMLLQVLQALPAQGESFTTL
ARELSARTFSRHVVQRQRVSGQVQALQNHYRKYLCLLASDAEVPEPCLPR
QYWEELGAPEALREQPWPLPVQMELGKLLAEMLVQATQMPCSLDKPHRSS
RLVPVLYHVYSFRNVQQIGILKPHPAYVQLLEKAAEPTLTFEAVDVPMLC
PPLPWTSPHSGAFLLSPTKLMRTVEGATQHQELLETCPPTALHGALDALT
QLGNCAWRVNGRVLDLVLQLFQAKGCPQLGVPAPPSEAPQPPEAHLPHSA
APARKAELRRELAHCQKVAREMHSLRAEALYRLSLAQHLRDRVFWLPHNM
DFRGRTYPCPPHFNHLGSDVARALLEFAQGRPLGPHGLDWLKIHLVNLTG
LKKREPLRKRLAFAEEVMDDILDSADQPLTGRKWWMGAEEPWQTLACCME
VANAVRASDPAAYVSHLPVHQDGSCNGLQHYAALGRDSVGAASVNLEPSD
VPQDVYSGVAAQVEVFRRQDAQRGMRVAQVLEGFITRKVVKQTVMTVVYG
VTRYGGRLQIEKRLRELSDFPQEFVWEASHYLVRQVFKSLQEMFSGTRAI
QHWLTESARLISHMGSVVEWVTPLGVPVIQPYRLDSKVKQIGGGIQSITY
THNGDISRKPNTRKQKNGFPPNFIHSLDSSHMMLTALHCYRKGLTFVSVH
DCYWTHAADVSVMNQVCREQFVRLHSEPILQDLSRFLVKRFCSEPQKILE
ASQLKETLQAVPKPGAFDLEQVKRSTYFFS.

### 3. Modified Mitochondrial DNA-Binding Polypeptides

The mitochondrial DNA binding polypeptide can be modified to include a PTD and optionally a targeting signal. The targeting signal can include a sequence of monomers that facilitates the localization of the molecule to a specific tissue, cell, or organelle. The monomers can be amino acids, nucleotide or nucleoside bases, or sugar groups such as glucose, galactose, and the like which form carbohydrate targeting signals.

A preferred modified mtDNA-binding polypeptide has at least 80, 85, 90, 95, 99 or more percent sequence identity to

(SEQ ID NO:1)

MARRRRRRRR RRRMAFLRSM WGVLSALGRS GAELCTGCGS
RLRSPFSFVY LPRWFSSVLA SCPKKPVSSY LRFSKEQLPI
FKAQNPDAKT TELIRRIAQR WRELPDSKKK IYQDAYRAEW
QVYKEEISRF KEQLTPSQIM SLEKEIMDKHLKRKAMTKKK
ELTLLGKPKR PRSAYNVYVA ERFQEAKGDS
PQEKLKTVKENWKNLSDSEK ELYIQHAKED ETRYHNEMKS
WEEQMIEVGR KDLLRRTIKKQRKYGAEEC.

Another embodiment provides a nucleic acid encoding the polypeptide according to SEQ ID NO: 1.

Still another embodiment provides a nucleic acid having at least 80, 85, 90, 95, 99 or more percent sequence identity to

(SEQ ID NO:25)

ATGGCGCGTCGTCGTCGTCGTCGTCGTCGTCGTCGTCGTATGGCGTTTCT
CCGAAGCATGTGGGGCGTGCTGAGTGCCCTGGGAAGGTCTGGAGCAGAGC
TGTGCACCGGCTGTGGAAGTCGACTGCGCTCCCCCTTCAGTTTTGTGTAT
TTACCGAGGTGGTTTTCATCTGTCTTGGCAAGTTGTCCAAAGAAACCTGT
AAGTTCTTACCTTCGATTTTCTAAAGAACAACTACCCATATTTAAAGCTC
AGAACCCAGATGCAAAAACTACAGAACTAATTAGAAGAATTGCCCAGCGT
TGGAGGGAACTTCCTGATTCAAAGAAAAAAATATATCAAGATGCTTATAG
GGCGGAGTGGCAGGTATATAAAGAAGAGATAAGCAGATTTAAAGAACAGC
TAACTCCAAGTCAGATTATGTCTTTGGAAAAAGAAAATCATGGACAAACA
TTTAAAAAGGAAAGCTATGACAAAAAAAAAAGAGTTAACACTGCTTGGAA
AACCAAAAAGACCTCGTTCAGCTTATAACGTTTATGTAGCTGAAAGATTC
CAAGAAGCTAAGGGTGATTCACCGCAGGAAAAGCTGAAGACTGTAAAGGA
AAACTGGAAAAATCTGTCTGACTCTGAAAAGGAATTATATATTCAGCATG
CTAAAGAGGACGAAACTCGTTATCATAATGAAATGAAGTCTTGGGAAGAA
CAAATGATTGAAGTTGGACGAAAGGATCTTCTACGTCGCACAATAAAGAA
ACAACGAAAATATGGTGCTGAGGAGTGTTAA.

The sequence encoding the protein transduction domain is underlined, and the sequence encoding the mitochondrial localization signal is double underline.

A. Protein Transduction Domain

The mtDNA-binding polypeptide can be modified to include a protein transduction domain (PTD), also known as cell penetrating peptides (CPPS). PTDs are known in the art, and include but are not limited to small regions of proteins that are able to cross a cell membrane in a receptor-independent mechanism (Kabouridis, P., Trends in Biotechnology (11):498-503 (2003)). Although several of PTDs have been documented, the two most commonly employed PTDs are derived from TAT (Frankel and Pabo, Cell, 55(6): 1189-93 (1988)) protein of HIV and Antennapedia transcription factor from Drosophila, whose PTD is known as Penetratin (Derossi et al., J Biol Chem., 269(14):10444-50 (1994)).

The Antennapedia homeodomain is 68 amino acid residues long and contains four alpha helices. Penetratin is an active domain of this protein which consists of a 16 amino acid sequence derived from the third helix of Antennapedia. TAT protein consists of 86 amino acids and is involved in the replication of HIV-1. The TAT PTD consists of an 11 amino acid sequence domain (residues 47 to 57; YCRKKRRQRRR (SEQ. ID. NO. 4)) of the parent protein that appears to be critical for uptake. Additionally, the basic domain Tat(49-57) or RKKRRQRRR (SEQ. ID NO. 5) has been shown to be a PTD. In the current literature TAT has been favored for fusion to proteins of interest for cellular import. Several modifications to TAT, including substitutions of Glutatmine to Alanine, i.e., Q→A, have demonstrated an increase in cellular uptake anywhere from 90% (Wender et al., Proc Natl Acad Sci USA., 97(24):13003-8 (2000)) to up to 33 fold in mammalian cells. (Ho et al., Cancer Res., 61(2):474-7 (2001)) The most efficient uptake of modified proteins was revealed by mutagenesis experiments of TAT-PTD, showing that an 11 arginine stretch was several orders of magnitude more efficient as an intercellular delivery vehicle. Thus, some embodiments include PTDs that are cationic or amphipathic. Additionally exemplary PTDs include but are not limited to poly-Arg—RRRRRRR (SEQ. ID. NO.: 7); PTD-5-RRQRRTSKLMKR (SEQ. ID. NO., 8); Transportan GWTLNSAGYLLGKINLKALAALAKKIL (SEQ. ID. NO.: 9); KALA—WEAKLAKALAKALAKHLAKALAKALKCEA (SEQ. ID. NO.: 10); and RQIKIWFQNRRMKWKK (SEQ. ID. NO.: 11).

B. Targeting Signal or Domain

In still other embodiments, the modified mtDNA-binding polypeptide is optionally modified to include a targeting signal or domain. The targeting signal or sequence can be specific for a host, tissue, organ, cell, organelle, non-nuclear organelle, or cellular compartment. For example, the compositions disclosed herein can be modified with galactosyl-terminating macromolecules to target the compositions to the liver or to liver cells. The modified compositions selectively enter hepatocytes after interaction of the carrier galactose residues with the asialoglycoprotein receptor present in large amounts and high affinity only on these cells. Moreover, the compositions disclosed here can be targeted to other specific intercellular regions, compartments, or cell types.

In one embodiment, the targeting signal binds to its ligand or receptor which is located on the surface of a target cell such as to bring the vector and cell membranes sufficiently close to each other to allow penetration of the vector into the cell. Additional embodiments of the present disclosure are directed to specifically delivering polynucleotides to specific tissue or cell types, wherein the polynucleotides can encode a polypeptide or interfere with the expression of a different polynucleotide. The polynucleotides delivered to the cell can encode polypeptides that can enhance or contribute to the functioning of the cell.

In a preferred embodiment, the targeting molecule is selected from the group consisting of an antibody or antigen binding fragment thereof, an antibody domain, an antigen, a T-cell receptor, a cell surface receptor, a cell surface adhesion molecule, a major histocompatibility locus protein, a viral envelope protein and a peptide selected by phage display that binds specifically to a defined cell.

Targeting polynucleotides to specific cells can be accomplished by modifying the disclosed compositions to express specific cell and tissue targeting signals. These sequences target specific cells and tissues, but in some embodiments the interaction of the targeting signal with the cell does not occur through a traditional receptor:ligand interaction. The eukaryotic cell comprises a number of distinct cell surface molecules. The structure and function of each molecule can be specific to the origin, expression, character and structure of the cell. Determining the unique cell surface complement of molecules of a specific cell type can be determined using techniques well known in the art.

One skilled in the art will appreciate that the tropism of the vector compositions described can be altered by merely changing the targeting signal. In one specific embodiment, compositions are provided that enable the addition of cell surface antigen specific antibodies to the vector for targeting the delivery of polynucleotides. Exemplary cell surface antigens are provided in Table 1 and described herein.

It is known in the art that nearly every cell type in a tissue in a mammalian organism possesses some unique cell surface receptor or antigen. Thus, it is possible to incorporate nearly any ligand for the cell surface receptor or antigen as a targeting signal. For example, peptidyl hormones can be used a targeting moieties to target delivery to those cells which possess receptors for such hormones. Chemokines and cytokines can similarly be employed as targeting signals to target delivery of the complex to their target cells. A variety of technologies have been developed to identify genes that are preferentially expressed in certain cells or cell states and one of skill in the art can employ such technology to identify targeting signals which are preferentially or uniquely expressed on the target tissue of interest

i. Brain Targeting

In one embodiment, the targeting signal is directed to cells of the nervous system, including the brain and peripheral nervous system. Cells in the brain include several types and states and possess unique cell surface molecules specific for the type. Furthermore, cell types and states can be further characterized and grouped by the presentation of common cell surface molecules.

In one embodiment, the targeting signal is directed to specific neurotransmitter receptors expressed on the surface of cells of the nervous system. The distribution of neurotransmitter receptors is well known in the art and one so skilled can direct the compositions described by using neurotransmitter receptor specific antibodies as targeting signals. Furthermore, given the tropism of neurotransmitters for their receptors, in one embodiment the targeting signal consists of a neurotransmitter or ligand capable of specifically binding to a neurotransmitter receptor.

In one embodiment, the targeting signal is specific to cells of the nervous system which may include astrocytes, microglia, neurons, oligodendrites and Schwann cells. These cells can be further divided by their function, location, shape, neurotransinitter class and pathological state. Cells of the nervous system can also be identified by their state of differentiation, for example stem cells. Exemplary markers specific for these cell types and states are well known in the art and include, but are not limited to CD 133 and Neurosphere.

ii. Muscle Targeting

In one embodiment, the targeting signal is directed to cells of the musculoskeletal system. Muscle cells include several types and possess unique cell surface molecules specific for the type and state. Furthermore, cell types and states can be further characterized and grouped by the presentation of common cell surface molecules.

In one embodiment, the targeting signal is directed to specific neurotransmitter receptors expressed on the surface of muscle cells. The distribution of neurotransmitter receptors is well known in the art and one so skilled can direct the compositions described by using neurotransmitter receptor specific antibodies as targeting signals. Furthermore, given the tropism of neurotransmitters for their receptors, in one embodiment the targeting signal consists of a neurotransmitter. Exemplary neurotransmitters expressed on muscle cells that can be targeted include but are not limited to acetycholine and norepinephrine.

In one embodiment, the targeting signal is specific to muscle cells which consist of two major groupings, Type I and Type II. These cells can be further divided by their function, location, shape, myoglobin content and pathological state. Muscle cells can also be identified by their state of differentiation, for example muscle stem cells. Exemplary markers specific for these cell types and states are well known in the art include, but are not limited to MyoD, Pax7 and MR4.

iii. Tumor Targeting

In one embodiment, the targeting signal is used to selectively target tumor cells. Tumor cells express cell surface markers which may only be expressed in the tumor or present in non tumor cells but preferentially presented in tumor cells. Exemplary tumor specific cell surface markers include, but are not limited to, alfa-fetoprotein (AFP), C-reactive protein (CRP), cancer antigen-50 (CA-50), cancer antigen-125 (CA-125) associated with ovarian cancer, cancer antigen 15-3 (CA15-3) associated with breast cancer, cancer antigen-9 (CA-19) and cancer antigen-242 associated with gastrointestinal cancers, carcinoembryonic antigen (CEA), carcinoma associated antigen (CAA), chromogranin A, epithelial mucin antigen (MC5), human epithelium specific antigen (HEA), Lewis(a) antigen, melanoma antigen, melanoma associated antigens 100, 25, and 150, mucin-like carcinoma-associated antigen, multidrug resistance related protein (MRPm6), multidrug resistance related protein (MRP41), Neu oncogene protein (C-erbB-2), neuron specific enolase ASE), P-glycoprotein (mdr1 gene product), multidrug-resistance-related antigen, p170, multidrug-resistance-related antigen, prostate specific antigen (PSA), CD56, and NCAM. In one embodiment, the targeting signal consists of antibodies which are specific to the tumor cell surface markers.

iv. Antibodies

Another embodiment provides an antibody or antigen binding fragment thereof bound to the disclosed recombinant polypeptides acting as the targeting signal. The antibodies or antigen binding fragment thereof are useful for directing the vector to a cell type or cell state. In one embodiment, the recombinant polypeptide possesses an antibody binding domain, for example from proteins known to bind antibodies such as Protein A and Protein G from Staphylococcus aureus. Other domains known to bind antibodies are known in the art and can be substituted. In certain embodiments, the antibody is polyclonal, monoclonal, linear, humanized, chimeric or a fragment thereof. Representative antibody fragments are those fragments that bind the antibody binding portion of the non-viral vector and include Fab, Fab′, F(ab′), Fv diabodies, linear antibodies, single chain antibodies and bispecific antibodies known in the art.

In some embodiments, the targeting domain includes all or part of an antibody that directs the vector to the desired target cell type or cell state. Antibodies can be monoclonal or polyclonal, but are preferably monoclonal. For human gene therapy purposes, antibodies are derived from human genes and are specific for cell surface markers, and are produced to reduce potential immunogenicity to a human host as is known in the art. For example, transgenic mice which contain the entire human immunoglobulin gene cluster are capable of producing “human” antibodies can be utilized. In one embodiment, fragments of such human antibodies are employed as targeting signals. In a preferred embodiment, single chain antibodies modeled on human antibodies are prepared in prokaryotic culture.

Additional embodiments of the present disclosure are directed to specifically delivering the polypeptide to intracellular compartments or organelles. Eukaryotic cells contain membrane bound structures or organelles. Organelles can have single or multiple membranes and exist in both plant and animal cells. Depending on the function of the organelle, the organelle can consist of specific components such as proteins and cofactors. The polypeptides delivered to the organelle can enhance or contribute to the functioning of the organelle. Some organelles, such as mitochondria and chloroplasts, contain their own genome. Nucleic acids are replicated, transcribed, and translated within these organelles. Proteins are imported and metabolites are exported. Thus, there is an exchange of material across the membranes of organelles. In some embodiments, mitochondrial polypeptides are specifically delivered to mitochondria.

Exemplary organelles include the nucleus, mitochondrion, chloroplast, lysosome, peroxisome, Golgi, endoplasmic reticulum, and nucleolus. Synthetic organelles can be formed from lipids and can contain specific proteins within the lipid membranes. Additionally, the content of synthetic organelles can be manipulated to contain components for the translation of nucleic acids.

v. Nuclear Localization Signals

The mtDNA-binding polypeptides disclosed herein can include one or more nuclear localization signals. Nuclear localization signals (NLS) or domains are known in the art and include for example, SV 40 T antigen or a fragment thereof, such as PKKKRKV (SEQ. ID. NO., 12). The NLS can be simple cationic sequences of about 4 to about 8 amino acids, or can be bipartite having two interdependent positively charged clusters separated by a mutation resistant linker region of about 10-12 amino acids. Additional representative NLS include but are not limited to GKKRSKV (SEQ. ID. NO.: 13); KSRKRKL (SEQ. ID. NO.: 14); KRPAATKKAGQAKKKKLDK (SEQ. ID. NO.: 15); RKKRKTEEESPLKDKAKKSK (SEQ. ID. NO.: 16); KDCVMNKHHRNRCQYCRLQR (SEQ. ID. NO.: 17); PAAKRVKLD (SEQ. ID. NO.: 18); and KKYENVVIKRSPRKRGRPRK (SEQ. ID. NO.: 19).

vi. Mitochondria Targeting

In other embodiments of the present disclosure, mtDNA-binding polypeptides are disclosed that specifically target mitochondria. Mitochondria contain the molecular machinery for the conversion of energy from the breakdown of glucose into adenosine triphosphate (ATP). The energy stored in the high energy phosphate bonds of ATP is then available to power cellular functions. Mitochondria are mostly protein, but some lipid, DNA and RNA are present. These generally spherical organelles have an outer membrane surrounding an inner membrane that folds (cristae) into a scaffolding for oxidative phosphorylation and electron transport enzymes. Most mitochondria have flat shelf-like cristae, but those in steroid secreting cells may have tubular cristae, The mitochondrial matrix contains the enzymes of the citric acid cycle, fatty acid oxidation and mitochondrial nucleic acids.

Mitochondrial DNA is double stranded and circular. Mitochondrial RNA comes in the three standard varieties; ribosomal, messenger and transfer, but each is specific to the mitochondria. Some protein synthesis occurs in the mitochondria on mitochondrial ribosomes that are different than cytoplasmic ribosomes. Other mitochondrial proteins are made on cytoplasmic ribosomes with a signal peptide that directs them to the mitochondria. The metabolic activity of the cell is related to the number of cristae and the number of mitochondria within a cell. Cells with high metabolic activity, such as heart muscle, have many well developed mitochondria. New mitochondria are formed from preexisting mitochondria when they grow and divide.

The inner membranes of mitochondria contain a family of proteins of related sequence and structure that transport various metabolites across the membrane. Their amino acid sequences have a tripartite structure, made up of three related sequences about 100 amino acids in length. The repeats of one carrier are related to those present in the others and several characteristic sequence features are conserved throughout the family.

Targeting of specific polypeptides to organelles can be accomplished by modifying the disclosed compositions to contain specific organelle targeting signals. These sequences target specific organelles, but in some embodiments the interaction of the targeting signal with the organelle does not occur through a traditional receptor:ligand interaction. The eukaryotic cell comprises a number of discrete membrane bound compartments, or organelles. The structure and function of each organelle is largely determined by its unique complement of constituent polypeptides. However, the vast majority of these polypeptides begin their synthesis in the cytoplasm. Thus organelle biogenesis and upkeep require that newly synthesized proteins can be accurately targeted to their appropriate compartment. This is often accomplished by amino-terminal signaling sequences, as well as post-translational modifications and secondary structure. For mitochondria, several amino-terminal targeting signals have been deduced and are known in the art.

In one embodiment, the organelle targeting signal can contain at least two, preferably 5-15, most preferably about 11 charged groups, causing the targeting signal to be drawn to organelles having a net opposite charge. In another embodiment, the targeting signal can contain a series of charged groups that cause the targeting signal to be transported into an organelle either against or down an electromagnetic potential gradient. Suitable charged groups are groups that are charged under intracellular conditions such as amino acids with charged functional groups, amino groups, nucleic acids, and the like. Mitochondrial localization/targeting signals generally consist of a leader sequence of highly positively charged amino acids. This allows the protein to be targeted to the highly negatively charged mitochondria. Unlike receptor:ligand approaches that rely upon stochastic Brownian motion for the ligand to approach the receptor, the mitochondrial localization signal of some embodiments is drawn to mitochondria because of charge.

In order to enter the mitochondria, a protein generally must interact with the mitochondrial import machinery, consisting of the Tim and Tom complexes (Translocase of the Inner/Outer Mitochondrial Membrane), With regard to the mitochondrial targeting signal, the positive charge draws the linked protein to the complexes and continues to draw the protein into the mitochondria. The Tim and Tom complexes allow the proteins to cross the membranes. Accordingly, one embodiment of the present disclosure delivers compositions of the present disclosure to the inner mitochondrial space utilizing a positively charged targeting signal and the mitochondrial import machinery. In another embodiment, PTD-linked polypeptides containing a mitochondrial localization signal do not seem to utilize the TOM/TIM complex for entry into the mitochondrial matrix, see Del Gaizo et al. Mol Genet Metab. 80(1-2):170-80 (2003).

Given the importance of mitochondria in human disease, cell proliferation, cell death, and aging, embodiments of the present disclosure also encompasses the manipulation of the mitochondrial function to supply the means by which known mitochondrial diseases (LHON, MELAS, etc.) and putative mitochondrial diseases (aging, Alzheimer's, Parkinson's, Diabetes, Heart Disease) can be treated.

vii. Chloroplast Targeting

In another embodiment, mtDNA-binding polypeptides disclosed herein specifically deliver polypeptides to chloroplasts by including a chloroplast localization signal or domain. For chloroplasts, several amino-terminal targeting signals have been deduced are known in the art. The chloroplast is a photosynthetic organelle in eukaryotes with a double surrounding membrane. The fluid inside the double-membrane is called the stroma. The chloroplast has a nucleoid region to house its circular, naked DNA. The stroma is also the site of the Calvin Cycle. The Calvin Cycle is the series of enzyme-catalyzed chemical reactions that produce carbohydrates and other compounds from carbon dioxide.

Within the stroma are tiny membrane sacs called thylakoids. The sacs are stacked in groups. Each group is called a granum. There are many grana in each chloroplast. The thylakoid membranes are the site of photosynthetic light reactions. The thylakoids have intrinsic and extrinsic proteins, some with special prosthetic groups, allowing for electrons to be moved from protein complex to protein complex. These proteins constitute an electron transport system sometimes known as the Z-scheme.

The prosthetic group for two critical membrane proteins (P680 and P700) is a chlorophyll a pigment molecule. These chlorophyll-binding proteins give the thylakoids an intense green color. The many thylakoids in a chloroplast give the chloroplast a green color. The many chloroplasts in a leaf mesophyll cell give that cell a green color. The many mesophyll cells in a leaf give the leaf a green color. The chlorophyll molecule absorbs light energy and an electron is boosted within the electron cloud in a resonating chemical structure surrounding a magnesium ion. This excited electron is removed by the surrounding electron transport proteins in the membrane. The movement of these electrons, and accompanying protons, results ultimately in the trapping of energy in a phosphate bond in ATP. The thylakoid is thus the location for light absorption and ATP synthesis. The stroma uses the ATP to store the trapped energy in carbon-carbon bonds of carbohydrates. Some chloroplasts show developing starch grains. These represent complex polymers of carbohydrates for long-term storage.

Given the bioenergetic functions of chloroplasts, the ability to introduce exogenous polypeptides may lead to plants with increased viability in otherwise hostile environments and increased efficiency of photosynthesis. Thus, other embodiments are directed to the modification of chloroplasts for more effective biosynthesis strategies for commercial compounds.

TABLE 1
Targeting Signals for Cell Types or Cell States.

| Cell Surface Antigen/ | |
|---|---|
| Cell Type | Cell Ligand |
| Airway cells | Surfactant proteins A and B |
| Arterial wall | Artery wall binding peptide |
| ASGP receptor | Asialoglycoproteins |
| ASGP receptor | Synthetic galactosylated ligands |
| Carbohydrates | Lectins |
| CD3 | Anti-CD 3 |
| CD5 | Anti-CD 5 |
| CD44 | hyaluronic acid fragments |
| CD117 | Steel factor, Anti CD117 |
| EGF-R | EGF, EGF peptide Anti EGF-R, |
| | TGF-alpha |
| ErbB2 | anti ErbB2 |
| FcR | IgG |
| FGF2-R | basic FGF |
| Folate receptor | Folate |
| Hepatocyte basolateral | Malarial circumsporozoite protein |
| surface | |
| Her2 | Anti HER2 |
| Insulin receptor | Insulin |
| Integrin | RGD peptide |
| LDL receptor family | Receptor associated protein (RAP) |
| (hepatocytes) | |
| Mannose receptor (macrophages) | Synthetic ligands, mannosylated |
| Nerve growth factor (NGF) | NGF serived synthetic peptide |
| receptor TrkA | |
| Neuroblastoma | Antibody ChCE7 |
| Ovarian carcinoma cell | Antibody OV-TL16 Fab′ fragment |
| surface antigen OA3 | |
| PECAM (lung endothelium) | anti-PECAM antibody |
| Poly-immunoglobulin receptor | Anti-secretory component |
| Serpin-enzyme receptor | peptide ligand |
| Surface immunoglobulin | Anti-IgG, Anti-idiotype |
| Thrombomodulin | Anti-thrombomodulin |
| Tn carbohydrate | Anti-Tn |
| Transferrin receptor | Transferrin |
| Airway cells | Surfactant proteins A and B |
| Arterial wall | Artery wall binding peptide |
| ASGP receptor | Asialoglycoproteins |
| ASGP receptor | Synthetic galactosylated ligands |
| Carbohydrates | Lectins |

### 4. Methods of Treatment

Embodiments of the present disclosure provide compositions and methods applicable to increasing mitochondrial biogenesis and oxidative metabolism. Cell dysfunction as a consequence of reduced mitochondrial biogenesis and/or oxidative metabolism can also be treated or reduced using the disclosed compositions and methods. It has been discovered that delivering recombinant mtDNA-binding proteins to mitochondria increases mitochondrial biogenesis and/or oxidative metabolism. In still another embodiment, the recombinant mtDNA-binding proteins serve as a vector for delivering polynucleotides to mitochondria.

Embodiments of the present disclosure provide compositions and methods applicable for therapeutic protocols and the treatment of gene related diseases or disorders where mitochondrial dysfunction is primary event or secondary to the cause. Cell mitochondrial dysfunction can also be treated or reduced using the disclosed compositions and methods. In particular, diseases producing mitochondrial dysfunction are specifically targeted. Diseases where improved mitochondrial function might prove therapeutic are also disclosed. The disease can be in children, for example individuals less that 18 years of age, typically less than 12 years of age, or adults, for example individuals 18 years of age or more. Thus, embodiments of the present disclosure are directed to treating a host diagnosed with a disease, in particular a genetic disease, by treating the host with a transducible protein capable of reducing mitochondrial dysfunction or improving mitochondrial function.

Suitable genetic based diseases that can be treated with the compositions disclosed herein include but are not limited to:

Mitochondrial Disease: Alpers Disease; Earth syndrome; β-oxidation defects; carnitine-acyl-carnitine deficiency; carnitine deficiency; co-enzyme Q10 deficiency; Complex I deficiency; Complex It deficiency; Complex III deficiency; Complex IV deficiency; Complex V deficiency; cytochrome c oxidase (COX) deficiency, LHON—Leber Hereditary Optic Neuropathy; MM—Mitochondrial Myopathy; LIMM—Lethal Infantile Mitochondrial Myopathy; MMC—Maternal Myopathy and Cardiomyopathy; NARP—Neurogenic muscle weakness, Ataxia, and Retinitis Pigmentosa; Leigh Disease; FICP—Fatal Infantile Cardiomyopathy Plus, a MELAS-associated cardiomyopathy; MELAS—Mitochondrial Encephalomyopathy with Lactic Acidosis and Strokelike episodes; LDYT—Leber's hereditary optic neuropathy and Dystonia; MERRF—Myoclonic Epilepsy and Ragged Red Muscle Fibers; MHCM—Maternally inherited Hypertrophic CardioMyopathy; CPEO—Chronic Progressive External Opthalmoplegia; KSS—Kearns Sayre Syndrome; DM—Diabetes Mellitus; DMDF Diabetes Mellitus+DeaFness; CIPO—Chronic Intestinal Pseudoobstruction with myopathy and Opthalmoplegia; DEAF—Maternally inherited DEAFness or aminoglycoside-induced DEAFness; PEM—Progressive encephalopathy; SNHL—SensoriNeural Hearing Loss; Enephalomyopathy; Mitoclhondrial cytopathy; Dilated Cardiomyopathy; GER—Gastrointestinal Reflux; DEMCHO—Dementia and Chorea; AMDF—Ataxia, Myoclonus; Exercise Intolerance; ESOC Epilepsy, Strokes, Optic atrophy, & Cognitive decline; FBSN Familial Bilateral Striatal Necrosis; FSGS Focal Segmental Glomerulosclerosis; LIMM Lethal Infantile Mitochondrial Myopathy; MDM Myopathy and Diabetes Mellitus; MEPR MyocIonic Epilepsy and Psychomotor Regression; MERME MERRF/MELAS overlap disease; MHCM Maternally Inherited Hypertrophic CardioMyopathy; MICM Maternally Inherited Cardiomyopathy; MILS Maternally Inherited Leigh Syndrome; Mitochondrial Encephalocardiomyopathy; Multisystem Mitochondrial Disorder (myopathy, encephalopathy, blindness, hearing loss, peripheral neuropathy); NAION Nonarteritic Anterior Ischemic Optic Neuropathy; NIDDM Non-Insulin Dependent Diabetes Mellitus; PEM Progressive Encephalopathy; PME Progressive Myoclonus Epilepsy; RTT Rett Syndrome; SIDS Sudden Infant Death Syndrome; MIDD Maternally Inherited Diabetes and Deafness; and MODY Maturity-Onset Diabetes of the Young, latrogenic Mitochondrial Dysfunction.

Nuclear Disease Muscular Dystrophies, Ellis-van Creveld syndrome, Marfan syndrome, Myotonic dystrophy, Spinal muscular atrophy, Achondroplasia, Amyotrophic lateral sclerosis, Charcot-Marie-Tooth syndrome, Cockayne syndrome, Diastrophic dysplasia, Duchenne muscular dystrophy, Ellis-van Creveld syndrome, Fibrodysplasia ossificans progressive, Alzheimer disease, Angelman syndrome, Epilepsy, Essential tremor, Fragile X syndrome, Friedreich's ataxia, Huntington disease, Niemann-Pick disease, Parkinson disease, Prader-Willi syndrome, Rett syndrome, Spinocerebellar atrophy, Williams syndrome, Ataxia telangiectasia, Anemia, sickle cell, Burkitt lymphoma, Gaucher disease, Hemophilia, Leukemia, Paroxysmal nocturnal hemoglobinuria, Porphyria, Thalassemia, Crohn's disease, Alpha-1-antitrypsin deficiency, Cystic fibrosis, Deafness, Pendred syndrome, Glaucoma, Gyrate atrophy of the choroid and retina, Adrenal hyperplasia, Adrenoleukodystrophy, Cockayne syndrome, Long QT syndrome, Immunodeficiency with hyper-IgM, Alport syndrome, Ellis-van Creveld syndrome, Fibrodysplasia ossificans progressive, Waardenburg syndrome, Werner syndrome.

Infectious Disease: Viral AIDS, AIDS Related Complex, Chickenpox (Varicella), Common cold, Cytomegalovirus Infection, Colorado tick fever, Dengue fever, Ebola haemorrhagic fever, Epidemic parotitis, Flu, Hand, foot and mouth disease, Hepatitis—Herpes simplex, Herpes zoster, HPV, Influenza, Lassa fever, Measles, Marburg haemorrhagic fever, Infectious mononucleosis, Mumps, Poliomyelitis, Progressive multifocal leukencephalopathy, Rabies, Rubella, SARS, Smallpox (Variola), Viral encephalitis, Viral gastroenteritis, Viral meningitis, Viral pneumonia, West Nile disease—Yellow fever; Bacterial—Anthrax, Bacterial Meningitis, Brucellosis, Bubonic plague, Campylobacteriosis, Cat Scratch Disease, Cholera, Diphtheria, Epidemic Typhus, Gonorrhea, Hansen's Disease, Legionellosis, Leprosy, Leptospirosis, Listeriosis, Lyme Disease, Melioidosis, MRSA infection, Nocardiosis, Pertussis, Pneumococcal pneumonia, Psittacosis, Q fever, Rocky Mountain Spotted Fever or RMSF, Salmonellosis, Scarlet Fever, Shigellosis, Syphilis, Tetanus, Trachoma, Tuberculosis, Tularemia, Typhoid Fever, Typhus, Whooping Cough; Parasitic—African trypanosomiasis, Amebiasis, Ascariasis, Babesiosis, Chagas Disease, Clonorchiasis, Cryptosporidiosis, Cysticercosis, Diphyllobothriasis, Dracunculiasis, Echinococcosis, Enterobiasis, Fascioliasis, Fasciolopsiasis, Filariasis, Free-living amebic infection, Giardiasis, Gnathostomiasis, Hymenolepiasis, Isosporiasis, Kala-azar, Leishmaniasis, Malaria, Metagonimiasis, Myiasis, Onchocerciasis, Pediculosis, Pinworm Infection, Scabies, Schistosomiasis, Taeniasis, Toxocariasis, Toxoplasmosis, Trichinellosis, Trichinosis, Trichuriasis, Trypanosomiasis, Pneumonia, Meningitis, Sepsis, Peritonitis, Arthritis (Infectious), Abscess, Carbuncle.

Cancers: Breast and ovarian cancer, Burkitt lymphoma, Chronic mycloid leukemia, Colon cancer, Lung cancer, Malignant melanoma, Multiple endocrine neoplasia, Neurofibromatosis, p53 LieFrauMeni, Pancreatic cancer, Prostate cancer, retinoblastoma, von Hippel-Lindau syndrome, Polycystic kidney disease, Tuberous sclerosis, liver cancer, brain cancer, gut cancers, bladder tumors.

Metabolic Disorders: Adrenoleukodystrophy, Atherosclerosis, Best disease, Gaucher disease, Glucose galactose malabsoiption, Gyrate atrophy, Juvenile onset diabetes, Obesity, Paroxysmal nocturnal hemoglobinuria, Phenylketonuria, Refsum disease, Tangier disease, Tay-Sachs disease, Adrenoleukodystrophy, Type 2 Diabetes, Gaucher disease, Hereditary hemochromatosis, Lesch-Nyhan syndrome, Maple syrup urine disease, Menkes syndrome, Niemann-Pick disease, Pancreatic cancer, Prader-Willi syndrome, Porphyria, Refsum disease, Tangier disease, Wilson's disease, Zellweger syndrome, progerias, SCID, dyslipidemia, steatohepatosis, fatty-liver disease.

Autoimmune Disorders: Autoimmune polyglandular syndrome, lupus, type I diabetes, scleroderma, multiple sclerosis, Crohn's disease, chronic active hepatitis, rheumatoid arthritis, Graves' disease, myasthenia gravis, myositis, antiphospholipid syndrome (APS), uveitis, polymyositis, Raynaud's phenomenon, and demyelinating neuropathies, and rare disorders such as polymyalgia rheumatica, temporal arteritis, Sjogren's syndrome, Behcet's disease, Churg-Strauss syndrome, and Takayasu's arteritis.

Inflammatory Disorders: Alopecia, Diastrophic dysplasia, Ellis-van Creveld syndrome, Asthma, Arthritis, including osteoarthritis, rheumatoid arthritis, and spondyloarthropathies.

Age-Related Disorders: Alzheimer disease, Parkinson's disease, atherosclerosis, age-related macular degeneration, age-related osteoporosis, sarcopenia, heart failure, heart attack, ischemia, mild cognitive impairment, atrophy, PAD, inflammation, diabetes, vascular disease, high blood pressure, hair graying, senility, dementia, actinic keratosis, seborrheic keratosis, dermal thinning, constipation, wrinkles, tremor, incontinence, presbyacusis, aneurysms, menopause, impotence, varicose veins, seborrheic dermatitis, teleangiectasia, lentigines, melanoma, purpura, pruritus, restless legs syndrome, insomnia, lipodystrophy, degenerative joint disease, myopathy, fronto-temporal dementia, Lewy body disease, neuropathy, migraine headache, chronic headache, heartburn, gastric and intestinal dismotility, gastroparesis, eyelid ptosis, atrial fibrillation, diastolic dysfunction, cataracts, blindness, chronic pain, fibromyalgia, hair loss, asthenia, abasia, gait dysfunction, vertigo.

Psychiatric Disorders: depression, mania, bipolar disorder, schizophrenia, autism spectrum disorders, narcolepsy, obsessive-compulsive disorder, hypersomnia, parasomnia.

The disclosed methods and compositions can also be used to treat, manage, or reduce symptoms associated with aging, in tissue regeneration/regenerative medicine, stem cell transplantation, cognitive enhancement, performance enhancement, and cosmetic alterations to human or non-human animals.

Embodiments of the present disclosure provide compositions and methods applicable to reducing obesity and/or increasing food intake. In certain embodiments the compositions can be used to promote weight loss in a subject without decreasing food intake relative to a control.

### 5. Administration

The compositions provided herein may be administered in a physiologically acceptable carrier to a host. Preferred methods of administration include systemic or direct administration to a cell. The compositions can be administered to a cell or patient, as is generally known in the art for protein therapies. One embodiment provides a pharmaceutical composition consisting essentially of a recombinant polypeptide having a polynucleotide-binding domain, a targeting domain, and a protein transduction domain and a pharmaceutically acceptable carrier or excipient. Preferably the polynucleotide-binding domain includes TFAM or a fragment thereof capable of binding a polynucleotide. The composition includes an effective amount of the recombinant polypeptide to increase mitochondrial metabolism.

The modified complex compositions can be combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as DETA; sugar alcohols such as mannitol or sorbitol; salt-forming coulnterions such as sodium; and/or nonionic surfactants such as Tween®, Pluronics® or PEG.

The compositions of the present disclosure can be administered parenterally. As used herein, “parenteral administration” is characterized by administering a pharmaceutical composition through a physical breach of a subject's tissue. Parenteral administration includes administering by injection, through a surgical incision, or through a tissue-penetrating non-surgical wound, and the like. In particular, parenteral administration includes subcutaneous, intraperitoneal, intravenous, intraarterial, intramuscular, intrasternal injection, and kidney dialytic infusion techniques.

Parenteral formulations can include the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Parenteral administration formulations include suspensions, solutions, emulsions in oily or aqueous vehicles, pastes, reconsitutable dry (i.e. powder or granular) formulations, and implantable sustained-release or biodegradable formulations. Such formulations may also include one or more additional ingredients including suspending, stabilizing, or dispersing agents. Parenteral formulations may be prepared, packaged, or sold in the form of a sterile injectable aqueous or oily suspension or solution. Parenteral formulations may also include dispersing agents, wetting agents, or suspending agents described herein. Methods for preparing these types of formulations are known. Sterile injectable formulations may be prepared using non-toxic parenterally-acceptable diluents or solvents, such as water, 1,3-butane diol, Ringer's solution, isotonic sodium chloride solution, and fixed oils such as synthetic monoglycerides or diglycerides. Other parentally-administrable formulations include microcrystalline forms, liposomal preparations, and biodegradable polymer systems. Compositions for sustained release or implantation may include pharmaceutically acceptable polymeric or hydrophobic materials such as emulsions, ion exchange resins, sparingly soluble polymers, and sparingly soluble salts.

Pharmaceutical compositions may be prepared, packaged, or sold in a buccal formulation. Such formulations may be in the form of tablets, powders, aerosols, atomized solutions, suspensions, or lozenges made using known methods, and may contain from about 0.1% to about 20% (w/w) active ingredient with the balance of the formulation containing an orally dissolvable or degradable composition and/or one or more additional ingredients as described herein. Preferably, powdered or aerosolized formulations have an average particle or droplet size ranging from about 0.1 nanometers to about 200 nanometers when dispersed.

As used herein, “additional ingredients” include one or more of the following: excipients, surface active agents, dispersing agents, inert diluents, granulating agents, disintegrating agents, binding agents, lubricating agents, sweetening agents, flavoring agents, coloring agents, preservatives, physiologically degradable compositions (e.g., gelatin), aqueous vehicles, aqueous solvents, oily vehicles and oily solvents, suspending agents, dispersing agents, wetting agents, emulsifying agents, demulcents, buffers, salts, thickening agents, fillers, emulsifying agents, antioxidants, antibiotics, antifungal agents, stabilizing agents, and pharmaceutically acceptable polymeric or hydrophobic materials. Other “additional ingredients” which may be included in the pharmaceutical compositions are known. Suitable additional ingredients are described in Remington's Pharmaceutical Sciences, Mack Publishing Co., Genaro, ed., Easton, Pa. (1985).

Dosages and desired concentrations modified vectors disclosed herein in pharmaceutical compositions of the present disclosure may vary depending on the particular use envisioned. The determination of the appropriate dosage or route of administration is well within the skill of an ordinary physician. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

### EXAMPLES

### Example 1

### Recombinant Constructs

The 11 amino acid protein transduction domain (PTD) consisting of 11 arginines was cloned in frame to the antibody binding portion, domain B, of Protein A from Staphylococcus aureus. The PTD-Domain B coding sequence was cloned in tandem upstream of the TFAM coding sequence and cloned into a bacterial expression vector. The recombinant protein was expressed in bacteria and isolated. Purified protein was concentrated and protein concentration was assessed with the Bradford Assay (Biorad). Purified protein was analyzed with SDS-Page to verify purity.

In a preferred embodiment, the recombinant polypeptide has at least 80, 85, 90, 95, 97, 99, or 100% sequence identity to

(SEQ ID NO:2)

MRRRRRRRRRRRGEGDIMGEWGNEIFGAIAGFLGGEMLSRAVCGTSRQLP
PVLGYLGSRQSSVLASCPKKPVSSYLRFSKEQLPIFKAQNPDAKTTELIR
RIAQRWRELPDSKKKIYQDAYRAEWQVYKE EISRFKEQLTPSQIMSLEK
EIMDKHLKRKAMTKKKELTLLGKPKRPRSAYNVYVALRFQEAKGDSPQEK
LKTVKENWKNLSDSEKELYIQHAKEDETRYHNEMKSWEEQMIEVGRKDLL
RRTIKKQRKYGAEEC

In another embodiment, the recombinant polypeptide is encoded by a nucleic acid having at least 80, 85, 90, 95, 97, 99, or 100% sequence identity to

(SEQ ID NO:3)

atgcggcgac gcagacgtcg tcgtcggcgg cgtcgcggcg
agggtgatat tatgggtgaa tgggggaacg aaattttcgg
agcgatcgct ggttttctcg gtggagaaat gttatcacgc
gcggtatgtg gcaccagcag gcagctgcct ccagtccttg
gctatctggg ttcccgccag tcatcggtgt tagcatcatg
tccgaaaaaa cctgtctcgt cgtacctgcg cttctccaaa
gagcagctgc cgatttttaa agcgcaaaat ccggatgcta
aaacgactga actgattcgc cgcattgcac aacgctggcg
cgaactcccg gacagtaaaa aaaaaattta tcaggacgcc
tatcgggctg aatggcaggt ctataaagag gagatctcac
gcttcaaaga acaattaacc ccgagtcaaa taatgtctct
ggaaaaagaa atcatggata aacacttaaa acgaaaggcg
atgacgaaga aaaaagaact gaccctgcta ggtaaaccta
agcgtccgcg ctctgcgtat aatgtgtacg tggcagaacg
ttttcaggag gccaaagggg attctccgca agaaaaactg
aagaccgtca aagaaaattg gaaaaacctg tctgatagcg
aaaaagaact gtacattcag cacgctaaag aagatgagac
gcggtatcac aacgaaatga aatcttggga agagcagatg
atcgaggtcg gtcggaagga tcttctccgt cgaaccatca
aaaaacagcg taaatatgga gcagaagagt gctga

### Example 2

### Construct Sequence Data

PTD-PA-TFAM (PTD solid underline; Tandem Domain B of Protein A Antibody Binding Domain double underline; TFAM dash underline) peptide Length (332):

(SEQ ID NO:20)
MRRRRRRRRRRRGEGDIMGEWGNEIFGAIAGFLGGEHDEAQQNAFYQVLNMPNLN
ADQRNGFIQSLKDDPSQSANVLGEAHDEAQQNAFYQVLNMPNLNADQRNGFIQSL
KDDPSQSANVLGEAGEG

[symbol]

[symbol]

[symbol]

[symbol]

[symbol]

Selected Model Organism Protein Similarities That Can Be Used In The Compositions And Methods Disclosed Herein:

### Organism, Protein And Percent Identity And Length Of Aligned Region

| | | |
|---|---|---|
| H. sapiens | sp: Q00059 - MTT1_HUMAN | 100%/246 aa |
| | Transcription factor 1, mitochondrial | (see ProtEST) |
| | precursor (MTTF1) | |
| M. musculus | ref: NP_033386.1 - transcription factor A, | 63%/237 aa |
| | mitochondrial [Mus musculus] | (see ProtEST) |
| R. norvegicus: | ref: NP_112616.1 - transcription factor A, mitochondrial | 64%/237 aa |
| | [Rattus norvegicus] | (see ProtEST) |
| A. thaliana | ref: NP_192846.1 - 98b like protein [Arabidopsis | 27%/189 aa |
| | thaliana] | (see ProtEST) |
| C. elegans | ref: NP_501245.1 - F45E4.9.p [Caenorhabditis | 27%/189 aa |
| | elegans] | (see ProtEST) |
| D. melanogaster: | ref: NP_524415.1 - mitochondrial transcription | 34%/183 aa |
| | factor A [Drosophila melanogaster] | (see ProtEST) |

### Example 3

### Mitochondrially Targeted Transducible TFAM Increases Mitochondrial Respiration

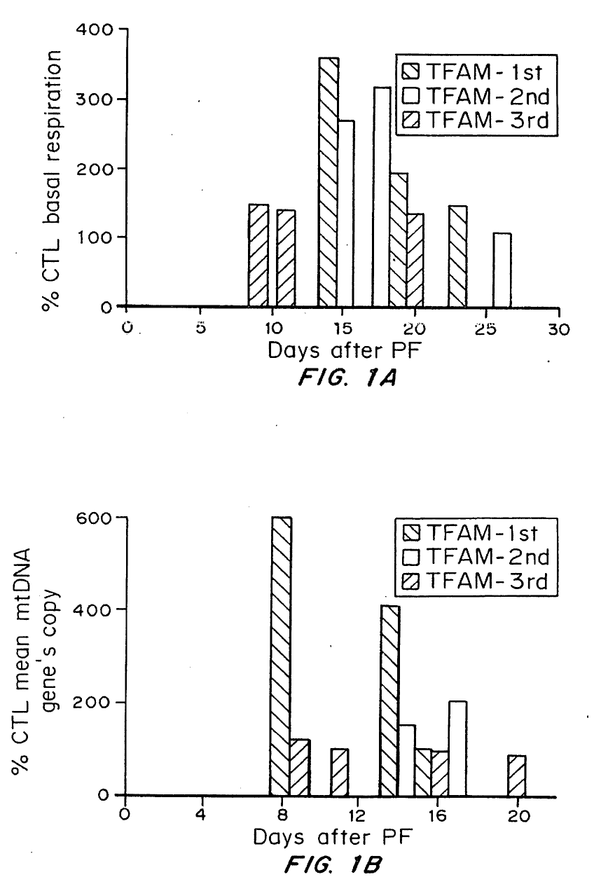

SH-SY5Y neuroblastoma cybrid cells carrying a G11778A mtDNA mutation in the ND4 gene from a patient afflicted with Leber's Hereditary Optic Neuropathy (LHON) were treated with mitochondrially targeted transducible TFAM (MTD-TFAM) or buffer control. MTD-TFAM is TFAM modified to include a protein transduction domain and a mitochondrial localization signal. Simultaneous “high-resolution” oximetry-respiration experiments using intact cells metabolizing glucose were conducted. The basal respiration values were depicted as a function of the same number of live cells expressed as a percentage of the corresponding buffer control cell values at each of the time points. Treatment with MTD-TFAM caused a time-dependent, reversible increase in basal respiration rates that reached a maximal ˜3-fold increase over control samples at around 2 weeks (FIGS. 1A-C).

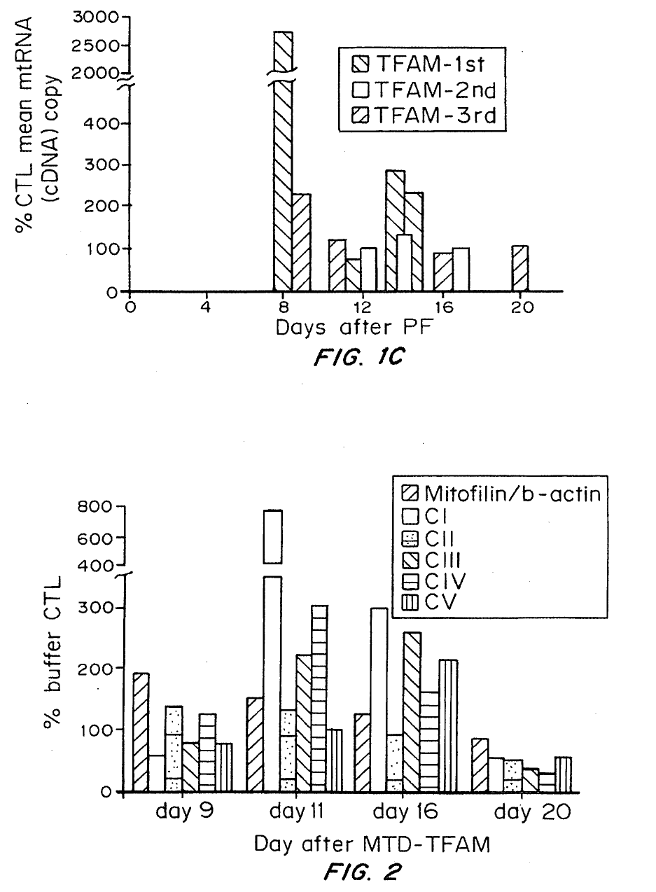

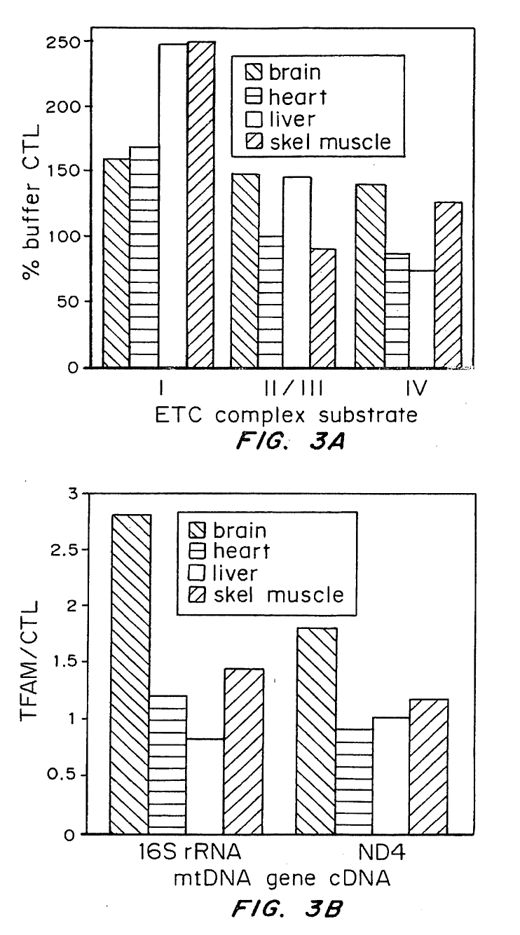

Because TFAM is a recognized essential factor for mitochondrial genome replication and transcription, MTD-TFAM exposure may be increasing mitochondrial gene replication, transcription and translation into respiratory proteins. Multiplex qPCR for several mitochondrial genes show substantial increases in mitochondrial gene copy numbers in both DNA and RNA (cDNA) following treatment with MTD-TFAM. Levels of multiple individual ETC proteins were assessed in treated cells using Western blots. Western blot analysis revealed that the relative mitochondrial mass in cells, expressed as a ratio of the outer mitochondrial membrane protein mitofilin to that of cytosolic beta actin, doubled (1.9-fold) in MTD-TFAM treated cells (FIG. 2). The levels of a mtDNA-encoded (CIV, subunit 2) and multiple nuclear genome-encoded ETC proteins from several complexes also increased substantially and reversibly in the MTD-TFAM treated cells with the greatest overall increases observed in complex I at day 11 (FIG. 2). The increase in respiration in cells was mirrored in animals. Normal adult male mice were treated with I.P. injections of MTD-TFAM or buffer control. Respiration was studied in mitochondrial preparations from brain, heart, skeletal muscle, kidney and liver. Treated mice showed increased respiration across tissues in the relative State 3 (+ADP) respiration rates for individual complexes (FIGS. 3A-3B). Thus the embodiments provided in the present disclosure enable methods to increase mitochondrial biogenesis and or mitochondrial oxidative metabolism.

### Example 4

### Mitochondrially Targeted Transducible TFAM Promotes Weight Loss

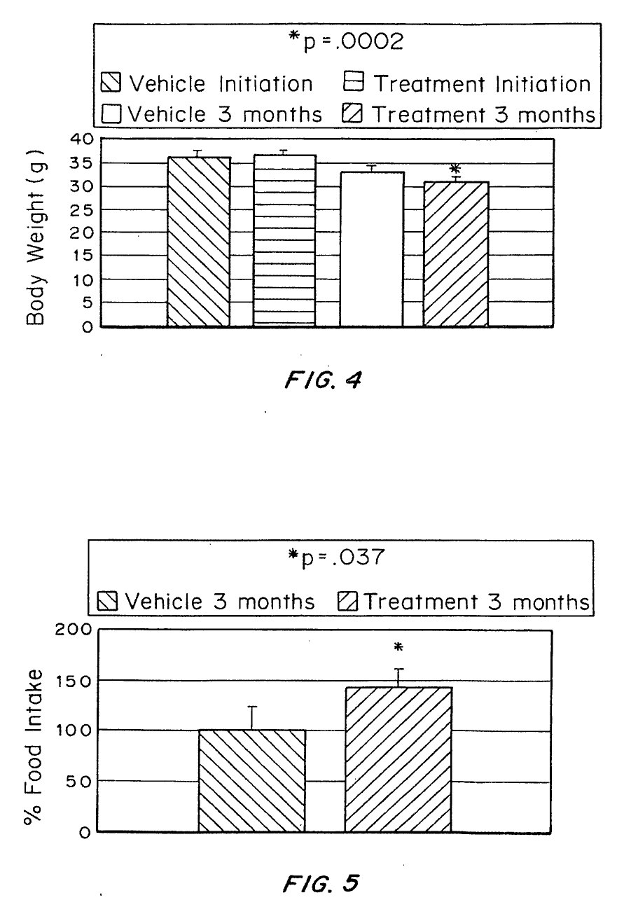

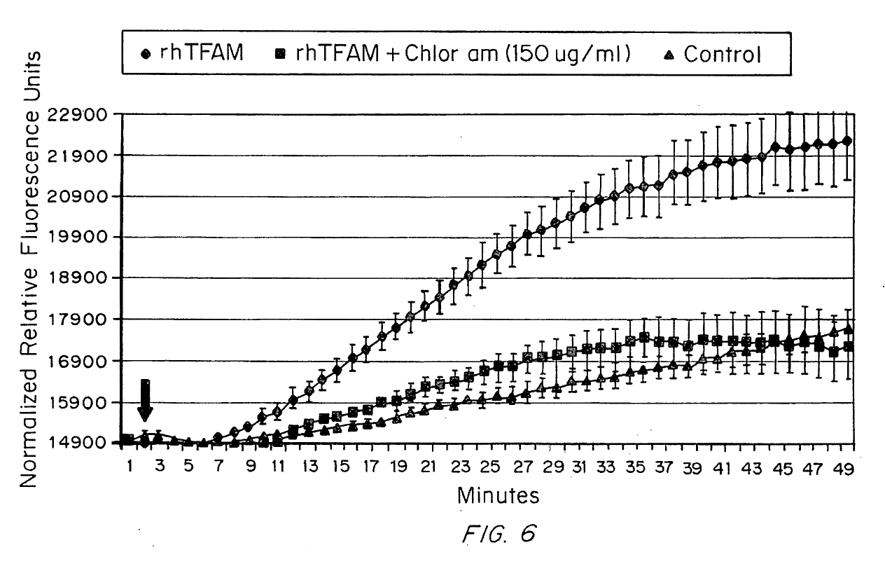

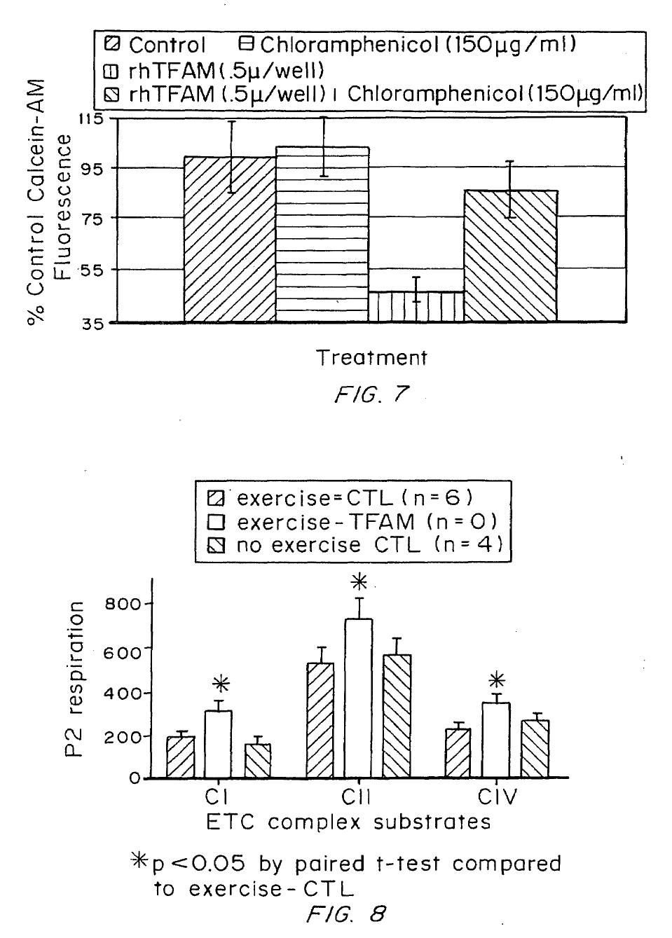

50 mice (25 vehicle control and 25 MTD-TFAM treated) were monitored for food intake and weight over a 3 month time interval during which saline or MTD-TFAM were injected IP on a monthly basis for a total of 3 injections, each. Mouse weight at the initiation of the study did not vary. Food intake was measured on a weekly basis. At the conclusion of the study, MTD-TFAM treated mice had statistically significant (p=0.0002) weight loss despite having consumed 43% more food than control animals over the course of the study (p=0.037) (FIGS. 4 and 5). In FIG. 5, 50 mice (25 vehicle control and 25 MTD-TFAM treated) were monitored for food intake over a 3 month time interval during which saline or MTD-TFAM were injected IP on a monthly basis for a total of 3 injections, each. Mouse weight at the initiation of the study did not vary. Food intake was measured on a weekly basis. At the conclusion of the study, MTD-TFAM treated mice had a statistically significant increase in food consumed of 43% over control animals over the course of the study (p=0.037). Thus, the disclosed methods can be used to treat obesity and/or increase food intake.

### Example 5

### Mitochondrially Targeted Transducible TEAM Induces Oxygen Consumption via Mitochondrial Transcription/Translation

50,000 FepG2 cells/well were cultured in BD OxygenBiosensor™ Plates with Cytodex-3 beads to enable attachment in normal DMEM with 10% FBS. Two hours before treatment with rhTFAM (0.6 U/well), a subset of wells were treated with chloramphenicol (1510 ug/ml) to block translation of mtDNA. The black arrow indicates addition of rhTFAM. Control cells were treated with vehicle. Over the next 50 minutes, fluorescence was assessed every minute according to manufacturer's protocol in a Tecan M200 plate reader. Intact HepG2 cells treated with rhTFAM consume significantly more oxygen than controls (p<0.001) within minutes expressed as normalized relative fluorescence units. RHTFAM stimulated oxygen consumption can be blocked by inhibiting translation of mtDNA encoded proteins of the electron transport chain, suggesting rhTFAM is increasing oxygen consumption by stimulating mtDNA transcription. The results are the combination of 3 separate experiments consisting of 12 wells per condition for an n=36. Error bars indicate standard deviation.

### Example 6

### Mitochondrially Targeted Transducible TFAM Reduces Cancer Metastatic Potential

The InnoCyte™ Laminin-Based 96-well Cell Invasion Assay was used to determine the invasive capacity of HepG2 cells treated with rhTFAM using laminin as a significant barrier to invasion. The assay is based on the Boyden-chamber principle. Where laminin layer forms an effective barrier that prevents non-invasive cells from passing through the 8 mm pores. Invasive cells that can degrade the laminin layer will migrate through the membrane and be detected with a fluorescent dye, Calcein-AM. An equal number of HepG2 cells plated in the 96-well cell invasion assay were treated with 0.5 U/well of rhTFAM. A subset of cells were pre-treated with chloramphenicol (150 ug/ml). Controls received vehicle alone. Twenty-four hours after rhTFAM treatment, invaded cells were dislodged according to manufacturer protocol and assayed with calcein-AM in a Tecan M200 plate reader. RHTFAM treatment reduced invasion of HepG2 cells by over 50% (p<0.003; n 12). This effect was blocked by chloramphenicol, suggesting mtDNA translation is required. Error bars indicate standard deviation.

### Example 7

### Mitochondrially Targeted Transducible TFAM Improves Brain Mitochondrial Activity Greater Than Exercise

Normal adult male C57BL/6 mice were injected via tail vein with rhTFAM (100u, n=6) or buffer vehicle control (n=6). Each mouse received one IV injection every week for a total of four injections. Before starting injections, each mouse was trained for endurance on a rotarod (Columbus Instruments). The training session consisted of two sessions of 120 sec at 5 rpm, two sessions of 120 sec at 10 rpm, two sessions of 120 sec at 15 rpm; there was a 10 minute rest period between each session. Each testing period consisted of three consecutive sessions at 15 rpm, followed by three consecutive sessions at 20 rpm for weeks 0-4, Because of increasing endurance, during weeks 3-4, three consecutive 30 rpm sessions were added after the 20 rpm sessions. There was a 10-minute rest period between each session; each session lasted no longer than 2000 sec. One week after the training regimen, brain mitochondrial P2 pellets were isolated and assayed for respiration fed substrates for the Complexes I, II and IV. Naïve, untrained mouse brain mitochondria were included for comparison purposes.

Sequence data for the sequences referenced herein are known in the art, for example in GenBank, and are incorporated by reference herein, in their entirety.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and the present disclosure and protected by the following claims.

What is claimed is:

1. A method for improving mitochondrial function comprising administering to a subject an effective amount of a mitochondrial transcription factor to increase mitochondrial respiration relative to a control.

2. The method of claim 1 wherein the mitochondrial transcription factor is recombinant.

3. The method of claim 1 wherein the mitochondrial transcription factor has at least 85% sequence identity to SEQ ID NO:6 (transcription factor A, mitochondrial (TFAM)).

4. The method of claim 1 wherein the mitochondrial transcription factor has at least 85% sequence identity to SEQ ID NO:22 transcription factor B1, mitochondrial (TFB1M)).

5. The method of claim 1 wherein the mitochondrial transcription factor has at least 85% sequence identity to SEQ ID NO:23 transcription factor B2, mitochondrial (TFB2M))

6. A method for improving mitochondrial function comprising administering to a subject an effective amount of polymerase (RNA) mitochondrial (DNA directed) (POLRMT) to increase mitochondrial respiration.

7. The method of claim 6 wherein the POLRMT has at least 85% sequence identity to SEQ ID NO:24.

8. A recombinant polypeptide comprising
a) a mitochondrial transcription factor;
b) a protein transduction domain operably linked to the mitochondrial transcription factor; and
c) a non-nuclear localization signal operably linked to mitochondrial transcription factor.

9. The recombinant polypeptide of claim 1, wherein the mitochondrial transcription factor is mitochondrial transcription factor A (TFAM).

10. The recombinant polypeptide of claim 2, wherein the mitochondrial transcription factor A comprises an amino acid sequence 95% identical to SEQ ID NO:6.

11. The recombinant polypeptide of claim 1, wherein the protein transduction domain is selected from the group consisting of to HIV TAT YGRKKRRQRRR (SEQ. ID NO.:4), RKKRRQRRR (SEQ. ID NO.:5); 7-15 positively charged amino acid residues, Penetratin, poly-Arg-RRRRRRR (SEQ. ID. NO.: 7); PTD-5-RRQRRTSKLMKR (SEQ. ID. NO.: 8); Transportan GWTLNSAGYLLGKINLKALAALAKKIL (SEQ. ID. NO.: 9); KALA-WEAKLAKALAKALAKHLAKALAKALKCEA (SEQ. ID. NO.: 10); and RQIKIWFQNRRMKWKK (SEQ, ID. NO.: 11).

12. The recombinant polypeptide of claim 1, wherein the mitochondrial transcription factor comprises human TFAM.

13. The recombinant polypeptide of claim 1 wherein the recombinant polypeptide comprises a conservative amino acid substitution.

14. The recombinant polypeptide of claim 1 wherein the non-nuclear localization signal comprises a mitochondrial localization signal.

15. A recombinant polypeptide encoded by a nucleic acid having at least 95% sequence identity to SEQ ID NO:3.

16. A recombinant polypeptide having an amino acid sequence according to SEQ ID NO:2.

17. A pharmaceutical composition comprising
a) a recombinant polypeptide comprising
i) a mitochondrial transcription factor A (TFAM) polypeptide or a variant thereof wherein the TFAM variant comprises one or more substitutions, additions, deletions relative to the TFAM polypeptide; and
ii) a protein transduction domain operably linked to the TFAM polypeptide or variant thereof; and
iii) a non-nuclear localization signal operably linked to the TFAM polypeptide or variant thereof; and
b) a pharmaceutically acceptable carrier.

18. The pharmaceutical compositions of claim 17 further comprising a polynucleotide capable of being expressed in mitochondria.

19. A recombinant polypeptide comprising
a) an amino acid sequence comprising amino acid number 63 to amino acid number 128 of SEQ ID NO:1;
b) a protein transduction domain operably linked to the recombinant polypeptide; and
c) a non-nuclear localization signal operably linked to the recombinant polypeptide.

20. The recombinant polypeptide of claim 19 further comprising an amino acid sequence comprising amino acid number 168 to amino acid number 229 of SEQ ID NO:1.

21. A recombinant polypeptide comprising
a) amino acid sequence comprising amino acid number 168 to amino acid number 229 of SEQ ID NO: 1;
b) a protein transduction domain operably linked to the recombinant polypeptide; and
c) a non-nuclear localization signal operably linked to the recombinant polypeptide.

22. The recombinant polypeptide of claim 21 further comprising an amino acid sequence comprising amino acid number 63 to amino acid number 128 of SEQ ID NO:1.

23. A recombinant polypeptide comprising
a) at least one HMG box domain wherein the HMG box domain comprises 75 amino acid residues, and wherein the 75 amino acid residues comprise highly conserved proline, aromatic and basic residues;
b) a protein transduction domain operably linked to the recombinant polypeptide; and
c) a non-nuclear localization signal operably linked to the recombinant polypeptide.

24. A non-viral vector comprising
a recombinant polypeptide comprising
a) at least two HMG box domains wherein the HMG box domains comprise 75 amino acid residues wherein the 75 amino acid residues comprise highly conserved proline, aromatic and basic residues;
b) a protein transduction domain operably linked to the recombinant polypeptide; and
c) a non-nuclear localization signal operably linked to the recombinant polypeptide.

25. A complex comprising the recombinant polypeptide of claim 1 and a polynucleotide, wherein the polypeptide is not covalently bound to the polynucleotide.

26. A method for treating a mitochondrial disease comprising administering to a subject an effective amount of the recombinant polypeptide of claim 1 to alleviate a symptom of the mitochondrial disease.

27. A method of treating a mitochondrial disease or disorder comprising administering to a subject an effective amount of a recombinant mitochondrial transcription factor to alleviate a symptom of the mitochondrial disease.
